(19) United States
(12) Patent Application Publication
(10) Pub. No.: US 2026/0337201 A1
(43) Pub. Date: Oct. 29, 2026

(54) PROCESS FOR PRODUCING CRYSTALLINE FORMS OF SEPIAPTERIN

(71) Applicant: PTC Therapeutics, Inc., Warren, NJ (US)

(72) Inventors: Yuping Qiu, Princeton Junction, NJ (US); Helge Alfred Reisch, Sarasota, FL (US); Seongwoo Hwang, Edison, NJ (US); Ming Li, Shanghai (CN); Chaoyi Deng, Shanghai (CN); Alan Hyde, Skillman, NJ (US)

(73) Assignee: PTC Therapeutics, Inc., Warren, NJ (US)

(21) Appl. No.: 19/641,694

(22) Filed: Apr. 8, 2026

Related U.S. Application Data
(60) Provisional application No. 63/791,292, filed on Apr. 18, 2025.

(51) Int. Cl. C07D 475/04

(52) U.S. Cl. CPC C07D 475/04

(57) ABSTRACT

The disclosure provides methods for making high-purity crystalline form F of sepiapterin free base suitable for scaleup up to a large-scale multi-kilogram batch, while establishing a minimum product purity. In particular, the disclosure provides a robust reproducible method for preparing large scale quantities of purified crystalline form F of sepiapterin free base comprising preparing a crystalline form of sepiapterin free base, comprising purifying crude sepiapterin free base through a purification cycle of salt formation, followed by free-basing to regenerate the sepiapterin free base in a pure form (purified sepiapterin free base), and subsequently recrystallizing the purified free base to obtain a specific crystalline polymorphic form.

### RELATED APPLICATIONS

This application claims priority to, and the benefit of, U.S. Provisional Patent Application No. 63/791,292, filed Apr. 18, 2025, the disclosure of which is incorporated herein by reference in its entirety.

### BACKGROUND OF THE INVENTION

Sepiapterin is a naturally occurring small molecule that is an intermediate substrate for de novo synthesis of tetrahydrobiopterin (BH₄) via the pterin salvage pathway. BH₄ is an essential cofactor, for a variety of oxidative enzymes including phenylalanine hydroxylase (PAH), tyrosine hydroxylase, tryptophan hydroxylase, fatty acid glyceryl ether oxygenase, and NO synthase. Therapeutic treatment with orally administered synthetic BH₄ (e.g., sapropterin dihydrochloride) is limited by its poor bioavailability. Specifically, its uptake by target cells is hampered by rapid oxidation to 7,8-dihydrobiopterin (BH₂) and poor membrane penetration. In contrast, sepiapterin is stable in plasma, actively transported into cells, and converted to intracellular BH₄ by a rapid unidirectional two-step enzymatic reduction process as part of the pterin salvage pathway.

Sepiapterin is susceptible to degradation in the presence of moisture, oxygen, and sunlight. Furthermore, many solid-state forms of sepiapterin suffer from poor process efficiency, flow characteristics, and stability, thereby limiting its production on a larger scale.

WO 2018/102315 (the '315 publication) discloses crystalline Form F of sepiapterin free base and a method for preparing Form F on a small scale, e.g., about 200 mg. The '315 publication, however, fails to provide any procedure for preparation of crystalline Form F on a large scale (e.g., multi-gram or multi-kilogram batch) or methods to ensure minimum product purity.

Thus, a need exists for specific crystalline forms having favorable physical properties and methods of preparation thereof that are suitable for scaleup to a multi-gram or multi-kilogram batch, while establishing a minimum product purity.

### SUMMARY OF THE INVENTION

Disclosed herein are novel methods for preparing purified crystalline forms of sepiapterin. In one aspect, the disclosure provides a reproducible method for preparing large scale quantities of purified crystalline form of sepiapterin comprising preparing a crystalline form of sepiapterin free base, comprising purifying crude sepiapterin free base through a purification cycle of salt formation, followed by free-basing to regenerate the sepiapterin free base in purified form (purified sepiapterin free base), and subsequently recrystallizing the purified free base to obtain a specific crystalline polymorphic form. A consistent process of generating specific crystalline polymorphic forms of sepiapterin, including polymorphic form F, is also described. The inventors have developed a robust, reproducible process for consistently producing large-scale quantities of high-purity crystalline Form F of sepiapterin free base.

In the first aspect, the disclosure provides a method of preparing crystalline form F of sepiapterin free base comprising:
(a) treating sepiapterin free base with an acid to obtain sepiapterin salt, and freebasing the salt to obtain a purified sepiapterin free base;
(b) thermocycling the purified sepiapterin free base from step (a) in one or more solvents to obtain sepiapterin free base Form D, Form F, or a mixture thereof; and
(c) filtering and drying the sepiapterin free base from step (b) to obtain sepiapterin free base Form F.

In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate, and sepiapterin sulfate. In some embodiments, the salt of sepiapterin is sepiapterin hydrochloride.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

In some embodiments, the base is selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, freebasing in step (a) is carried out at a temperature of less than 25 °C.

In some embodiments, thermocycling in step (b) comprises heating sepiapterin free base in one or more solvents to a temperature from 25 °C to about 75 °C followed by cooling to a temperature of less than about 25 °C In some embodiments, thermocycling comprises heating to at least about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling comprises heating to a temperature of at least 35 °C followed by cooling to a temperature of less than 25 °C, about 20 °C, about 15 °C, about 10 °C, about 5 °C, about 0 °C, about −5 °C, about −10 °C, or intermediate temperatures therein. In some embodiments, the thermocycling in step (b) comprises heating the sepiapterin free base in one or more solvents from step (b) to a temperature of from about 30 °C to about 70 °C followed by cooling to a temperature of from about −2 °C to about 15 °C In some embodiments, the thermocycling comprises heating the sepiapterin free base from step (b) to about 45 °C then cooling to about 5 °C.

In some embodiments, sepiapterin freebase from step (b) comprises sepiapterin Form D, Form F, or mixtures thereof. In some embodiments, sepiapterin freebase from step (b) comprises sepiapterin Form D. In some embodiments, sepiapterin freebase from step (b) comprises sepiapterin Form F. In some embodiments, sepiapterin free base form from step (b) does not include sepiapterin free base Form E in an amount detectable by X-ray powder diffraction.

In some embodiments, there is an inverse relationship between the heating temperature and heating time. For example, a higher heating temperature may require a shorter heating time, and a lower heating temperature may require a longer heating time to produce sepiapterin Form D and/or Form F.

In some embodiments, step (b) further comprises adding seed crystals of sepiapterin free base to facilitate crystallization of sepiapterin free base Form D and/or Form F. In some embodiments, the seed crystals include crystalline Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, seed crystals of sepiapterin free base Form D, and/or F are added in step (b) to facilitate the production of sepiapterin free base Form D. In some embodiments, the method does not include addition of seed crystals of sepiapterin free base. In some embodiments, the seed crystals are added before freebasing

In some embodiments, the one or more solvents in step (b) are selected from the group consisting of water, ethanol, acetone, isopropyl alcohol, 2-butanone (methyl ethyl ketone/MEK), tetrahydrofuran (THF), 2-methyltetrahydrofuran (MeTHF), N,N-dimethylacetamide (DMAc), dimethyl sulfoxide (DMSO), N,N-dimethylacetamide (DMF), and mixtures thereof.

In some embodiments, the one or more solvents in step (b) comprise water, acetone, or mixtures thereof. In some embodiments, the one or more solvents in step (b) is a mixture of acetone, water, and DMSO. In some embodiments, the one or more solvents in step (b) comprise acetone and water in a water:acetone vol/vol ratio of from 1:0 to 0:1. In some embodiments, the one or more solvents in step (b) is a mixture of water and acetone in a water:acetone vol/vol ratio of about 4:1. In some embodiments, the one or more solvents in step (b) is a mixture of water, and MEK in a water:MEK vol/vol ratio of from 1:0 to 0:1. In some embodiments, the one or more solvents in step (b) is a mixture of water, and MEK in a water:MEK vol/vol ratio of from 4:1 to 1:0. In some embodiments, the one or more solvents in step (b) is a mixture of water and THF in a water:THF vol/vol ratio of from 1:0 to 0:1. In some embodiments, the one or more solvents in step (b) is a mixture of water and THF in a water:THF vol/vol ratio of from 4:1 to 1:0. In some embodiments, the one or more solvents in step (b) is a mixture of water and MeTHF in a water:MeTHF vol/vol ratio of from 1:0 to 0:1. In some embodiments, the one or more solvents in step (b) is a mixture of water and MeTHF in a water:MeTHF vol/vol ratio of from 4:1 to 1:0. In some embodiments, the one or more solvents in step (b) is a mixture of water and ethanol in a water:ethanol vol/vol ratio of from 1:0 to 0:1. In some embodiments, the one or more solvents in step (b) is a mixture of water and ethanol in a water:ethanol vol/vol ratio of from 4:1 to 1:0. In some embodiments, the one or more solvents in step (b) is a mixture of water and IPA in a water:IPA vol/vol ratio of from 1:0 to 0:1. In some embodiments, the one or more solvents in step (b) is a mixture of water and IPA in a water:IPA vol/vol ratio of from 4:1 to 1:0. In some embodiments, the one or more solvents in step (b) is a mixture of water and DMAc in a water:DMAc vol/vol ratio of from 1:0 to 0:1. In some embodiments, the one or more solvents in step (b) is a mixture of water and DMAc in a water:DMAc vol/vol ratio of from 3:1 to 1:0. In some embodiments, the one or more solvents in step (b) is IPA.

In some embodiments, step (b) comprises the one or more solvents in an amount of at least about 7.5 volume equivalents (solvent volume relative to weight of sepiapterin salt, e.g., mL/g or L/kg)). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents.

In some embodiments, the one or more solvents in step (b) is a mixture of water and acetone. In some embodiments, step (b) comprises a mixture of water and acetone in an amount of at least about 7.5 volume equivalents (solvent volume relative to weight of sepiapterin salt). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water and acetone.

In some embodiments, the one or more solvents in step (b) is water. In some embodiments, step (b) comprises at least 7.5 volume equivalents of water. In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volumes therein of water.

In some embodiments, the one or more solvents in step (b) is acetone. In some embodiments, step (b) comprises at least 7.5 volume equivalents of acetone (solvent volume relative to weight of sepiapterin salt). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volumes therein of acetone.

In some embodiments, step (b) comprises a mixture of water, acetone, and DMSO in an amount of at least about 7.5 volume equivalents (solvent volume relative to weight of sepiapterin salt). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water, acetone, and DMSO.

In some embodiments, the one or more solvents in step (b) comprise water and DMSO in an amount of at least about 7.5 volume equivalents (solvent volume relative to weight of sepiapterin salt). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water, and DMSO.

In some embodiments, step (c) comprises filtering the free base Form D from step (b) and heating the filtered free base to a temperature of from about 20 °C to about 75 °C In some embodiments, the filtered free base in step (c) is dried via heating to a temperature of up to about 75 °C, about 70 °C, about 65 °C, about 60 °C, about 55 °C, about 50 °C, about 45 °C, about 40 °C, about 35 °C, about 30 °C, about 25 °C, about 20 °C, or any intermediate temperatures therein. In some embodiments, there is an inverse relationship between the time required for drying and the temperature required for drying. That is, a higher drying temperature may require a shorter drying time, and a lower drying temperature may require a longer drying time to produce sepiapterin free base Form F. In some embodiments, the drying is performed at a higher temperature for a shorter time. In some embodiments, the drying is performed at a lower temperature for a longer time. In some embodiments, the drying is monitored by Karl Fisher (KF) titration (oven method) of sepiapterin. In some embodiments, the drying is monitored to prevent the formation of sepiapterin free base Form G.

In some embodiments, the drying occurs at a temperature of at least about 20 °C, about 25 °C, about 30 °C, about 35 °C, about 40 °C, about 45 °C, about 50 °C, about 55 °C, about 60 °C, about 60 °C, about 70 °C, about 75 °C, or intermediate temperatures therein. In some embodiments, the drying temperature and time depends upon the water content in the filtered solids. For example, filtered solids with less water content will require a lower drying temperature and shorter time compared to filtered solids with higher water content. In some embodiments, the mixture is filtered and dried under vacuum (at a pressure lower than the atmospheric pressure). In some embodiments, step (c) comprises filtering and drying at 1 atmosphere pressure. In some embodiments, the water content from the filtered mixture depends upon the amount of solids, agitation speed, and the vacuum pressure.

In some embodiments, drying in step (c) comprises drying under vacuum. In some embodiments, drying in step (c) comprises drying under vacuum (from about −0.09 MPa to about −0.1 MPa) at a temperature of up to about 25 °C In some embodiments, drying in step (c) comprises drying under vacuum at a first temperature followed by drying under vacuum at a higher temperature. In some embodiments, drying in step (c) comprises drying under vacuum at a temperature of up to about 25 °C followed by drying under vacuum to a temperature of about 45 °C.

In some embodiments, steps (a), (b), and (c) are purged/degassed with an inert gas to minimize oxidative degradation of sepiapterin free base or a form thereof. In some embodiments, the inert gas is nitrogen, helium, or argon.

In some embodiments, the one or more solvents in step (b) are degassed with an inert gas (e.g., degassed by bubbling nitrogen).

In some embodiments, the drying is monitored by Karl Fischer (KF) titration (oven method) of sepiapterin. In some embodiments, the drying is maintained such that the KF value remains below about 5%. In some embodiments, the drying is maintained, such that the KF value remains below about 4.5%. In some embodiments, the drying is maintained such that the KF value remains at about 4%. In some embodiments, the drying is maintained, such that the KF value remains at about 3.5%. In some embodiments, the drying is maintained such that the KF titration value is maintained at a level to prevent over-drying of crystalline Form F of sepiapterin free base to form crystalline Form G of sepiapterin free base.

In some embodiments, sepiapterin free base Form D is washed with acetone prior to step (c). In some embodiments, the acetone is purged with inert gas, e.g., nitrogen, argon, or helium.

In some embodiments, the purity of the sepiapterin free base after drying is a chemical purity (i.e., the purity of sepiapterin to the exclusion of other chemical compounds, such as lactoylpterin). In some embodiments, the chemical purity is determined quantitatively. In some embodiments, the chemical purity is determined by HPLC.

In some embodiments, the sepiapterin free base after drying is qualitatively pure Form F, e.g., as determined by obtaining an X-ray powder diffraction (XRPD) pattern. In not contain peaks characteristic of other crystalline forms of sepiapterin free base (e.g., of Form A, Form B, Form C, Form D, Form E, and/or Form G). In some embodiments, an X-ray powder diffraction pattern of the sepiapterin free base after drying does not contain peaks characteristic of crystalline Form G. In some embodiments, an X-ray powder diffraction pattern of the sepiapterin free base after drying does not contain peaks characteristic of crystalline Form D.

In another aspect, the disclosure provides a method for preparing crystalline form F of sepiapterin free base comprising:
(a) treating sepiapterin free base with an acid to obtain sepiapterin salt, and freebasing the sepiapterin salt to obtain a purified sepiapterin free base;
(b) thermocycling the purified sepiapterin free base from step (a) in one or more solvents to obtain sepiapterin free base Form A, Form D, Form F, or a mixture thereof,
(c) optionally, thermocycling the sepiapterin free base from step (b) in one or more solvents to obtain sepiapterin free base Form D, Form F, or mixtures thereof, and
(d) filtering and drying the sepiapterin free base from step (b) or the optional step (c) to obtain sepiapterin free base Form F.

In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate, and sepiapterin sulfate. In some embodiments, the salt of sepiapterin is sepiapterin hydrochloride. In some embodiments, sepiapterin free base form after thermocycling includes sepiapterin Form D, and optionally, Form A and/or Form F.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

In some embodiments, the base is selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, freebasing in step (a) is carried out at a temperature of less than 25 °C.

In some embodiments, sepiapterin free base form before thermocycling comprises sepiapterin Form A. In some embodiments, sepiapterin free base form after first or second thermocycling includes sepiapterin Form D, and optionally, Form A, and/or Form F.

In some embodiments, the sepiapterin free base from step (b) is sepiapterin free base Form D. In some embodiments, the sepiapterin free base from step (b) is a mixture of sepiapterin free base Form A and sepiapterin free base Form D. In some embodiments, the sepiapterin free base is a mixture of sepiapterin free base Form A, Form D, and Form F. In some embodiments, the sepiapterin free base is a mixture of sepiapterin free base Form D, and Form F.

In some embodiments, the sepiapterin free base from step (c) is sepiapterin Form D. In some embodiments, the sepiapterin free base from step (c) is a mixture of sepiapterin Form D and sepiapterin Form F. In some embodiments, sepiapterin free base form from step (b) and optional step (c) does not include sepiapterin free base Form E in an amount detectable by X-ray powder diffraction.

In some embodiments, the sepiapterin free base is filtered in step (b) and resuspended in one or more solvents in step (c). In some embodiments, the sepiapterin free base is not filtered prior to step (c). In some embodiments, the sepiapterin free base in one or more solvents from step (b) is thermocycled directly in step (c) without prior filtration. In some embodiments, the one or more solvents in step (b) and optional step (c) are water and acetone.

In some embodiments, the thermocycling in step (b) and optional step (c) comprises heating sepiapterin free base in one or more solvents to a temperature of from 25 °C to about 75 °C followed by cooling to a temperature of less than about 25 °C In some embodiments, thermocycling comprises heating to at least about 25 °C, about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling comprises heating to a temperature of at least 35 °C followed by cooling to a temperature of less than about 25 °C, about 20 °C, about 15 °C, about 10 °C, about 5 °C, about 0 °C, about −5 °C, about −10 °C, or intermediate temperatures therein. In some embodiments, the thermocycling in step (b) and optional step (c) comprises heating the sepiapterin free base in one or more solvents from step (b) or optional step (c) to a temperature of from about 30 °C to about 75 °C or higher followed by cooling to a temperature of from about −5 °C to about 15 °C In some embodiments, the thermocycling comprises heating the sepiapterin free base from step (b) and/or optional step (c) to about 45 °C. then cooling to about 5 °C.

In some embodiments, there is an inverse relationship between the heating temperature and the heating time. For example, a higher heating temperature may require a shorter heating time, and a lower heating temperature may require a longer heating time to produce crystalline Form D and/or Form F.

In some embodiments, step (b) and optional step (c) further comprise adding seed crystals of sepiapterin free base to facilitate crystallization of sepiapterin free base Form D. In some embodiments, the seed crystals include Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, seed crystals of sepiapterin free base Form A, Form D, and/or Form F are added in step (b) and optional step (c) to facilitate the production of sepiapterin free base Form D. In some embodiments, the method does not include addition of seed crystals of sepiapterin free base.

In some embodiments, the one or more solvents in step (b) and optional step (c) are selected from the group consisting of water, ethanol, acetone, isopropyl alcohol, 2-butanone (methyl ethyl ketone/MEK), tetrahydrofuran (THF), 2-methyltetrahydrofuran (MeTHF), N,N-dimethylacetamide (DMAc), dimethyl sulfoxide (DMSO), N,N-dimethylacetamide (DMF), and mixtures thereof.

In some embodiments, the one or more solvents in step (b), and/or step (c) comprise water, acetone, or mixtures thereof. In some embodiments, the one or more solvents in step (b) and optional step (c) is a mixture of acetone and water. In some embodiments, the one or more solvents in step (b) and optional step (c) is a mixture of acetone, water and DMSO.

In some embodiments, the one or more solvents in step (b) and optional step (c) comprise water, acetone, or mixtures thereof. In some embodiments, the one or more solvents in step (b) and optional step (c) comprise water and acetone in a water:acetone vol/vol ratio of from 1:0 to 0:1. In some embodiments, the one or more solvents in step (b) and optional step (c) comprise water and acetone in a water:acetone vol/vol ratio of about 4:1 (1:0.25).

In some embodiments, step (b) and optional step (c) comprises the one or more solvents in an amount of at least about 7.5 volume equivalents (solvent volume relative to weight of sepiapterin salt, e.g., mL/gm or L/kg). In some embodiments, step (b) and optional step (c) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents. In some embodiments, the one or more solvents in step (b) and/or step (c) is a mixture of water and acetone. In some embodiments, step (b) and/or step (c) comprises a mixture of water and acetone in an amount of least about 7.5 volume equivalents (solvent volume relative to weight of sepiapterin salt). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water and acetone.

In some embodiments, sepiapterin free base is washed with acetone prior to step (d). In some embodiments, the acetone is purged with inert gas, e.g., nitrogen, argon, or helium.
some embodiments, the X-ray powder diffraction pattern of the sepiapterin free base after drying does

In some embodiments, step (d) comprises filtering the free base from step (b) or optional step (c) and heating the filtered free base to a temperature of from about 20 °C to about 75 °C In some embodiments, the filtered free base in step (d) is dried via heating to a temperature of up to about 75 °C, about 70 °C, about 65 °C, about 60 °C, about 55 °C, about 50 °C, about 45 °C, about 40 °C, about 35 °C, about 30 °C, about 25 °C, about 20 °C, or any intermediate temperatures therein.

In some embodiments, there is an inverse relationship between the time required for drying and the temperature required for drying. That is, a higher drying temperature may require a shorter drying time, and a lower drying temperature may require a longer drying time to produce sepiapterin free base Form F. In some embodiments, the drying is performed at a higher temperature for a shorter time. In some embodiments, the drying is performed at a lower temperature for a longer time. In some embodiments, the drying is monitored by Karl Fisher (KF) titration. In some embodiments, the drying is monitored to prevent the formation of crystalline Form G of sepiapterin free base.

In some embodiments, the drying occurs at a temperature of at least about 20 °C, about 25 °C, about 30 °C, about 35 °C, about 40 °C, about 45 °C, about 50 °C, about 55 °C, about 60 °C, about 60 °C, about 70 °C, about 75 °C, or intermediate temperatures therein. In some embodiments, the drying temperature and time depend upon the water content in the filtered solids. For example, filtered solids with less water content will require a lower drying temperature and shorter time compared to filtered solids with higher water content. In some embodiments, the mixture is filtered under vacuum (at a pressure lower than the surrounding atmospheric pressure). In some embodiments, step (d) comprises filtering and drying at 1 atmosphere pressure. In some embodiments, the water content from the filtered mixture depends upon the amounts of solids, agitation speed, and the vacuum pressure.

In some embodiments, the drying step (d) comprises drying under vacuum (from about −0.09 MPa to about −0.1 MPa). In some embodiments, the drying step (d) comprises drying under vacuum at a first temperature followed by drying under vacuum at a higher temperature. In some embodiments, the drying step (c) comprises drying under vacuum to a temperature of about 25 °C. followed by heating under vacuum to a temperature of about 45 °C In some embodiments, step (d) comprises drying at 1 atmosphere pressure.

In some embodiments, the entire process is purged/degassed with an inert gas to minimize oxidative degradation of sepiapterin free base or a form thereof. In some embodiments, the inert gas is nitrogen, helium, or argon.

In some embodiments, the one or more solvents in steps (a), (b), and optional step (c) are degassed with an inert gas (e.g., degassed by bubbling nitrogen).

In some embodiments, the drying is monitored by Karl Fischer (KF) titration (oven method) of sepiapterin. In some embodiments, the drying is maintained such that the KF value remains below about 5%. In some embodiments, the drying is maintained, such that the KF value remains below about 4.5%. In some embodiments, the drying is maintained such that the KF value remains at about 4%. In some embodiments, the drying is maintained, such that the KF value remains at about 3.5%. In some embodiments, the drying is maintained such that the KF titration value is maintained at a level to prevent over-drying of sepiapterin free base Form F to sepiapterin free base Form G.

In some embodiments, the purity of the sepiapterin free base after drying is a chemical purity (i.e., the purity of sepiapterin to the exclusion of other chemical compounds, such as lactoylpterin). In some embodiments, the chemical purity is determined quantitatively. In some embodiments, the chemical purity is determined by HPLC.

In some embodiments, the sepiapterin free base after drying is qualitatively pure Form F, e.g., as determined by obtaining an X-ray powder diffraction pattern. In some embodiments, the X-ray powder diffraction pattern of the sepiapterin free base after drying does not contain peaks characteristic of other crystalline forms of sepiapterin free base (e.g., of Form A, Form B, Form C, Form D, Form E and/or Form G). In some embodiments, an X-ray powder diffraction pattern of the sepiapterin free base after drying does not contain peaks characteristic of crystalline Form G. In some embodiments, an X-ray powder diffraction pattern of the sepiapterin free base after drying does not contain peaks characteristic of crystalline Form D.

In another aspect, the disclosure provides a method of preparing crystalline Form F of sepiapterin free base comprising:
(a) treating sepiapterin free base with an acid to obtain sepiapterin salt, and freebasing the salt to obtain purified sepiapterin free base;
(b) thermocycling the purified sepiapterin free base from step (a) in one or more solvents to obtain sepiapterin free base Form D, Form F, or a mixture thereof;
(c) filtering the sepiapterin free base from step (b) to obtain a wet cake comprising sepiapterin free base Form D, Form F, or a mixture thereof, and
(d) reslurrying the wet cake from step (c) and drying in a dry solvent to obtain sepiapterin free base Form F.

In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate, and sepiapterin sulfate. In some embodiments, the salt of sepiapterin is sepiapterin hydrochloride. In some embodiments, sepiapterin free base form after thermocycling includes sepiapterin Form D, and optionally, Form A and/or Form F.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

In some embodiments, the base is selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, freebasing in step (a) is carried out at a temperature of less than 25 °C.

In some embodiments, the sepiapterin free base form before thermocycling comprises sepiapterin Form A. In some embodiments, the sepiapterin free base form after thermocycling includes sepiapterin Form D, and optionally, Form A and/or Form F. In some embodiments, sepiapterin free base form from step (b) does not include sepiapterin free base Form E in an amount detectable by X-ray powder diffraction.

In some embodiments, thermocycling in step (b) comprises heating sepiapterin free base in one or more solvents to a temperature of from about 25 °C to about 75 °C followed by cooling to a temperature of less than about 25 °C In some embodiments, thermocycling comprises heating to at least about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling comprises heating from about 40 °C to about 55 °C, followed by cooling to a temperature of less than about 25 °C In some embodiments, thermocycling comprises heating from about 40 °C to about 55 °C followed by cooling to a temperature of from about −2 °C to about 15 °C In some embodiments, thermocycling comprises heating to a temperature of at least 35 °C followed by cooling to a temperature of less than 25 °C, about 20 °C, about 15 °C, about 10 °C, about 5 °C, about 0 °C, about −5 °C, about −10 °C, or intermediate temperatures therein. In some embodiments, the thermocycling in step (b) comprises heating the sepiapterin free base in one or more solvents from step (b) to a temperature of from about 30 °C to about 70 °C. followed by cooling to a temperature of from about −2 °C to about 15 °C In some embodiments, the thermocycling comprises heating the sepiapterin free base from step (b) to about 45 °C then cooling to about 5 °C.

In some embodiments, there is an inverse relationship between the heating temperature and heating time. For example, a higher heating temperature may require a shorter heating time, and a lower heating temperature may require a longer heating time to produce crystalline Form D and/or Form F.

In some embodiments, step (b) further comprises adding seed crystals of sepiapterin free base to facilitate crystallization of sepiapterin free base Form D and/or Form F. In some embodiments, the seed crystals include Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, seed crystals of sepiapterin free base Form A, Form D, and/or F are added in step (b) to facilitate the production of sepiapterin free base Form D. In some embodiments, the method does not include addition of seed crystals of sepiapterin free base.

In some embodiments, the one or more solvents in step (b) are selected from the group consisting of water, ethanol, acetone, isopropyl alcohol, 2-butanone (methyl ethyl ketone/MEK), tetrahydrofuran (THF), 2-methyltetrahydrofuran (MeTHF), N,N-dimethylacetamide (DMAc), dimethyl sulfoxide (DMSO), N,N-dimethylacetamide (DMF), and mixtures thereof.

In some embodiments, the one or more solvents in step (b) comprise water, acetone, or mixtures thereof. In some embodiments, the one or more solvents in step (b) is a mixture of acetone, water, and DMSO. In some embodiments, the one or more solvents in step (b) comprise acetone and water in a water:acetone vol/vol ratio of from 1:0 to 0:1. In some embodiments, the one or more solvents in step (b) is a mixture of water and acetone in a water:acetone vol/vol ratio of 4:1. In some embodiments, the one or more solvents in step (b) is a mixture of water and MEK in a water:MEK vol/vol ratio of from 1:0 to 0:1. In some embodiments, the one or more solvents in step (b) is a mixture of water and MEK in a water:MEK vol/vol ratio of from 4:1 to 1:0. In some embodiments, the one or more solvents in step (b) is a mixture of water and THF in a water:THF vol/vol ratio of from 1:0 to 0:1. In some embodiments, the one or more solvents in step (b) is a mixture of water and THF in a water:THF vol/vol ratio of from 4:1 to 1:0. In some embodiments, the one or more solvents in step (b) is a mixture of water and MeTHF in a water:MeTHF vol/vol ratio of from 1:0 to 0:1. In some embodiments, the one or more solvents in step (b) is a mixture of water and MeTHF in a water:MeTHF vol/vol ratio of from 4:1 to 1:0. In some embodiments, the one or more solvents in step (b) is a mixture of water and ethanol in a water:ethanol vol/vol ratio of from 1:0 to 0:1. In some embodiments, the one or more solvents in step (b) is a mixture of water and ethanol in a water:ethanol vol/vol ratio of from 4:1 to 1:0. In some embodiments, the one or more solvents in step (b) is a mixture of water and IPA in a water:IPA vol/vol ratio of from 1:0 to 0:1. In some embodiments, the one or more solvents in step (b) is a mixture of water and IPA in a water:IPA vol/vol ratio of from 4:1 to 1:0. In some embodiments, the one or more solvents in step (b) is a mixture of water and DMAc in a water:DMAc vol/vol ratio of from 1:0 to 0:1. In some embodiments, the one or more solvents in step (b) is a mixture of water and DMAc in a water:DMAc vol/vol ratio of from 3:1 to 1:0. In some embodiments, the one or more solvents in step (b) is IPA.

In some embodiments, step (b) comprises the one or more solvents in an amount of at least about 7.5 volume equivalents (solvent volume relative to weight of sepiapterin salt, e.g., mL/g or L/kg). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents.

In some embodiments, the one or more solvents in step (b) is a mixture of water and acetone. In some embodiments, step (b) comprises a mixture of water and acetone in an amount of at least about 7.5 volume equivalents (solvent volume relative to weight of sepiapterin salt). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water and acetone.

In some embodiments, the dry solvent in step (d) is selected from the group consisting of ethanol, acetone, 1-propanol, isopropyl alcohol, acetonitrile (MeCN), 2-butanone (MEK), methyl isobutyl ketone (MIBK), isopropyl acetate (IPAc), toluene, tetrahydrofuran (THF), 2-methyltetrahydrofuran (MeTHF), and mixtures thereof.

In some embodiments, the purity of the sepiapterin free base in step (d) is a chemical purity (i.e., the purity of sepiapterin to the exclusion of other chemical compounds, such as lactoylpterin). In some embodiments, the chemical purity is determined quantitatively. In some embodiments, the chemical purity is determined by HPLC. In some embodiments, the sepiapterin free base from step (d) is qualitatively pure Form F, e.g., as determined by obtaining an X-ray powder diffraction pattern. In some embodiments, the X-ray powder diffraction pattern of the sepiapterin free base from step (d) does not contain peaks characteristic of other crystalline forms of sepiapterin free base (e.g., of Form A, Form B, Form C, Form D, Form E, and/or Form G). In some embodiments, an X-ray powder diffraction pattern of the sepiapterin free base from step (d) does not contain peaks characteristic of crystalline Form G. In some embodiments, an X-ray powder diffraction pattern of the sepiapterin free base from step (d) does not contain peaks characteristic of crystalline Form D.

In some embodiments, the entire process is purged/degassed with an inert gas to minimize oxidative degradation of sepiapterin free base or a form thereof. In some embodiments, the inert gas is nitrogen, helium, or argon.

In some embodiments, the crystalline Form D is characterized by an X-ray powder diffraction pattern obtained by irradiation with Cu Kα X-rays having characteristic peaks expressed in degrees 2θ at about 8.9°, about 10.3°, and about 26.0°. In some embodiments, the crystalline Form D is characterized by an X-ray powder diffraction pattern obtained by irradiation with Cu Kα X-rays having peaks expressed in degrees 2θ at about 8.9°, about 10.3°, about 10.9°, about 17.8°, about 24.9°, about 26.0°, about 26.7°, about 26.8°, and about 28.3°.

In some embodiments, the crystalline Form F is characterized by an X-ray powder diffraction pattern obtained by irradiation with Cu Kα X-rays having characteristic peaks expressed in degrees 2θ at about 9.7°, about 10.2°, and about 11.3°. In some embodiments, the crystalline Form F is characterized by an X-ray powder diffraction pattern obtained by irradiation with Cu Kα X-rays having peaks expressed in degrees 2θ at about 9.7°, about 10.2°, about 11.3°, about 14.0°, about 14.6°, about 19.9°, about 22.2°, about 25.3°, and about 32.4°.

Additional characterization of the final sepiapterin free base Form F may be conducted beyond crystal form confirmation by XRPD. This may include assessing the appearance of the sepiapterin free base Form F, identifying the sepiapterin free base Form F through Raman spectroscopy, and confirming the HPLC retention time of the sample matches that of the reference. The process may also involve evaluating the purity of the sepiapterin free base Form F using high-performance liquid chromatography (HPLC), measuring residuals on ignition, analyzing residual solvents via gas chromatography with headspace sampling (GC-H), determining the particle size of the sepiapterin free base Form F, and conducting microbial tests on the sepiapterin free base Form F.

In another aspect, the disclosure provides a method of preparing sepiapterin free base Form F comprising:
(a) treating sepiapterin free base with an acid to obtain a sepiapterin salt, and freebasing the salt to obtain a purified sepiapterin free base; and
(b) thermocycling the purified sepiapterin free base from step (a) and drying in one or more dry solvents to obtain sepiapterin free base Form F, and optionally Form G.

In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate, and sepiapterin sulfate. In some embodiments, the salt of sepiapterin is sepiapterin hydrochloride. In some embodiments, sepiapterin free base form after thermocycling includes sepiapterin Form D, and optionally, Form A and/or Form F.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

In some embodiments, the base is selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, the sepiapterin free base form before thermocycling comprises sepiapterin Form A. In some embodiments, sepiapterin free base form after thermocycling includes sepiapterin Form F.

In some embodiments, thermocycling in step (b) comprises heating sepiapterin free base in one or more dry solvents to a temperature of from about 25 °C to about 75 °C followed by cooling to a temperature of less than 25 °C In some embodiments, thermocycling comprises heating to at least about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling comprises heating from about 40 °C to about 55 °C followed by cooling to a temperature of less than about 25 °C In some embodiments, thermocycling comprises heating from about 40 °C to about 55 °C followed by cooling to a temperature of from about −2 °C to about 15 °C In some embodiments, thermocycling comprises heating to a temperature of at least 35 °C followed by cooling to a temperature of less than 25 °C, about 20 °C, about 15 °C, about 10 °C, about 5 °C, about 0 °C, about −5 °C, about −10 °C, or intermediate temperatures therein. In some embodiments, the thermocycling in step (b) comprises heating the sepiapterin free base in one or more solvents from step (b) to a temperature of from about 30 °C to about 70 °C. followed by cooling to a temperature of from about −2 °C to about 15 °C In some embodiments, the thermocycling comprises heating the sepiapterin free base from step (b) to about 45 °C then cooling to about 5 °C.

In some embodiments, there is an inverse relationship between the heating temperature and heating time. For example, a higher heating temperature may require a shorter heating time, and a lower heating temperature may require a longer heating time to produce sepiapterin Form F.

In some embodiments, step (b) further comprises adding seed crystals of sepiapterin free base to facilitate crystallization of sepiapterin free base Form F. In some embodiments, the seed crystals include Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, seed crystals of sepiapterin free base Form D, and/or F are added in step (b) to facilitate the production of sepiapterin free base Form F. In some embodiments, the method does not include addition of seed crystals of sepiapterin free base.

In some embodiments, the one or more dry solvents in step (b) are selected from the group consisting of acetone, 2-butanone, methyl isobutyl ketone, isopropyl alcohol, dimethyl sulfoxide, and mixtures thereof.

In some embodiments, the one or more dry solvents in step (b) are isopropyl alcohol, acetone, or a mixture of acetone and DMSO.

In some embodiments, step (b) comprises the one or more dry solvents in an amount of at least about 7.5 volume equivalents (solvent volume relative to weight of sepiapterin salt, e.g., mL/g or L/kg)). In some embodiments, step (b) comprises about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents.

In another aspect, the disclosure provides a method for preparing sepiapterin Form F comprising:
(a) treating sepiapterin free base with an acid to obtain a sepiapterin salt, and freebasing the salt to obtain a purified sepiapterin free base;
(b) thermocycling the purified sepiapterin free base from step (a) in one or more solvents to obtain sepiapterin free base Form D, and optionally, Form F, and
(c) filtering and drying the sepiapterin free base from step (b) to obtain sepiapterin free base Form F,
wherein, thermocycling of purified sepiapterin free base from step (a) does not provide sepiapterin free base Form E in an amount detectable by X-ray powder diffraction.

In some embodiments, purified sepiapterin free base from step (a) does not include lactoylpterin % LC impurity in an amount of less than 1%.

In some embodiments, the sepiapterin free base from step (c) is qualitatively pure Form F, e.g., as determined by obtaining an X-ray powder diffraction pattern. In some embodiments, the X-ray powder diffraction pattern of the sepiapterin free base from step (c) does not contain peaks characteristic of other crystalline forms of sepiapterin free base (e.g., of Form A, Form B, Form C, Form D, Form E, and/or Form G). In some embodiments, an X-ray powder diffraction pattern of the sepiapterin free base from step (d) does not contain peaks characteristic of crystalline Form G. In some embodiments, an X-ray powder diffraction pattern of the sepiapterin free base from step (d) does not contain peaks characteristic of crystalline Form D.

In some embodiments, the entire process is purged/degassed with an inert gas to minimize oxidative degradation of sepiapterin free base or a form thereof. In some embodiments, the inert gas is nitrogen, helium, or argon.

In another aspect, the disclosure provides a method for preparing sepiapterin form D comprising:
(a) treating sepiapterin free base with an acid to obtain sepiapterin salt, and freebasing the salt to obtain a purified sepiapterin free base; and
(b) thermocycling the purified sepiapterin free base from step (a) in one or more solvents, to obtain sepiapterin free base Form D, and optionally Form F,
wherein freebasing in step (a) is carried out at a temperature of less than 25 °C, and
wherein thermocycling of purified sepiapterin free base from step (a) does not provide sepiapterin free base Form E in an amount detectable by X-ray powder diffraction.

In still another aspect, the disclosure provides a method for preparing sepiapterin form D comprising:
(a) treating sepiapterin free base with an acid to obtain sepiapterin salt, and freebasing the salt to obtain a purified sepiapterin free base; and
(b) thermocycling the purified sepiapterin free base from step (a) in one or more solvents to obtain sepiapterin free base Form D, and optionally Form F,
wherein sepiapterin salt in step (a) includes lactoylpterin % LC impurity in an amount of less than 1%, and
wherein thermocycling of purified sepiapterin free base from step (a) does not provide sepiapterin free base Form E in an amount detectable by X-ray powder diffraction.

In some embodiments, the crystalline Form D is characterized by an X-ray powder diffraction pattern obtained by irradiation with Cu Kα X-rays having characteristic peaks expressed in degrees 2θ at about 8.9°, about 10.3°, and about 26.0°. In some embodiments, the crystalline Form D is characterized by an X-ray powder diffraction pattern obtained by irradiation with Cu Kα X-rays having peaks expressed in degrees 2θ at about 8.9°, about 10.3°, about 10.9°, about 17.8°, about 24.9°, about 26.0°, about 26.7°, about 26.8°, and about 28.3°.

In some embodiments, the crystalline Form F is characterized by an X-ray powder diffraction pattern obtained by irradiation with Cu Kα X-rays having characteristic peaks expressed in degrees 2θ at about 9.7°, about 10.2°, and about 11.3°. In some embodiments, the crystalline Form F is characterized by an X-ray powder diffraction pattern obtained by irradiation with Cu Kα X-rays having peaks expressed in degrees 2θ at about 9.7°, about 10.2°, about 11.3°, about 14.0°, about 14.6°, about 19.9°, about 22.2°, about 25.3°, and about 32.4°.

Additional characterization of the final sepiapterin free base Form F may be conducted beyond crystal form confirmation by XRPD. This may include assessing the appearance of the sepiapterin free base Form F, identifying the sepiapterin free base Form F through Raman spectroscopy, and confirming the HPLC retention time of the sample matches that of the reference. The process may also involve evaluating the purity of the sepiapterin free base Form F using high-performance liquid chromatography (HPLC), measuring residuals on ignition, analyzing residual solvents via gas chromatography with headspace sampling (GC-H), determining the particle size of the sepiapterin free base Form F, and conducting microbial tests on the sepiapterin free base Form F.

### DEFINITIONS

As used herein and in the claims, the singular forms “a, “an,” and “the,” are intended to include the plural forms as well, unless the context clearly indicates otherwise. As used herein, when used in conjunction with the term “comprising” in the claims and/or in the specification can mean “one,” “one or more,” “at least one,” or “one or more than one.

Still further, the terms “having”, “including”, “containing” and “comprising” are interchangeable and one of skill in the art is cognizant that these terms are open ended terms.

It will be further understood that the terms “comprises”, “comprising”, “includes”, and/or “including,” when used herein, specify the presence of stated features, integers, steps, operations, elements, and/or components, but do not preclude the presence or addition of one or more other features, integers, steps, operations, elements, components, and/or groups thereof.

As used herein and in the claims, the term “consisting of” is a close ended term limiting the scope to only the explicitly listed elements and excluding any additional elements.

As used herein and in the claims, the phrase “at least one”, in reference to a list of one or more elements, should be understood to mean at least one element selected from any one or more of the elements in the list of elements, but not necessarily including at least one of each and every element specifically listed within the list of elements, and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

As used herein and in the claims, the term “about” when used in conjunction with a numerical value modifies the numerical value above and below the stated value by a variance of 10%. “About” and “approximately” shall generally mean an acceptable degree of error for the quantity measured given the nature or precision of the measurements. Exemplary degrees of error are within 20%, typically, within 10%, and more typically, within 5% of a given value or range of values.

The term “about” when used in conjunction with a numerical range, modifies the range by extending the boundaries on both ends of the range by a variance of 10%.

As used herein and in the claims, the term “BH₄ related disorder”, refers to any disease or disorder that may derive a therapeutic benefit from modulation (e.g., inhibition) of the level of tetrahydrobiopterin (BH₄), e.g., phenylketonuria.

As used herein and the claims, the term “dry solvent”, refers to an organic solvent containing less than 0.5% water.

As used interchangeably herein and in the claims, the terms “degassed”, “nitrogen purging”, and “inert gas purging” refer to a process that uses an inert gas to displace or remove unwanted gases, vapors, moisture, or contaminants from within a confined system or a vessel. In some embodiments, inert gas is used to purge reaction medium, including overhead space in a reactor or reaction container, a reaction mixture, solvent or a solution to displace dissolved oxygen or other dissolved gaseous contaminants. In some embodiments, nitrogen is used as an inert gas. Methods to degas solvents are known in the art. For example, a solvent may be degassed by “nitrogen purging/bubbling nitrogen” whereby a stream of nitrogen gas is passed through a solvent or solution to sufficiently purge the solution of dissolved gaseous contaminants.

As used interchangeably herein and in the claims, the terms “filter”, “filtered”, “filtering”, “filtration”, and the like refer to a process where a solid-liquid suspension or a mixture is passed through a filter, e.g., a cloth or a glass frit, to separate the solids from the liquids.

As used interchangeably herein and in the claims, the terms “deliquor” and “fully deliquored”, refer to the step after filtration wherein vacuum is applied to the initially filtered solids to remove most (>95%) of the residual solvent present to provide a “wet cake”.

As used herein and in the claims, the term “vacuum’ refers to a state where the pressure within a closed system is lower than the surrounding atmospheric pressure. It is a negative gauge pressure from about −0.09 to −0.1 MPa (megapascals), signifying that the pressure is lower than the ambient environment.

As used interchangeably herein and in the claims, the terms “mixing”, and “aging” refer to agitation of a reaction mixture or solution containing sepiapterin or a form thereof. In some embodiments, agitation is continuous. In some embodiments, the reaction mixture may be agitated such that there is no agglomeration of solids in the reaction mixture or solution. In some embodiments, agitation includes the use of an overhead stirrer. In some embodiments, agitation includes the use of a magnetic stir bar. In some embodiments, agitation includes continuous flow mixing elements (e.g., a T-mixer, a Y-mixer, or a static mixer).

When a range of values is listed herein, it is intended to encompass each value and sub-range within that range. For example, “1-5 g” or a range of “1 g to 5 g” is intended to encompass 1 g, 2 g, 3 g, 4 g, 5 g, 1-2 g, 1-3 g, 1-4 g, 1-5 g, 2-3 g, 2-4 g, 2-5 g, 3-4 g, 3-5 g, and 4-5 g.

As used interchangeably herein and in the claims, the terms “thermocycling” or “temperature cycling” refer to a change of temperature from a lower temperature to a higher temperature, followed by a return to a lower temperature. In some embodiments, the return to a lower temperature is to the same temperature as the initial lower temperature. In some embodiments, the return to a lower temperature is to a different (e.g., higher or lower) temperature as compared to the initial lower temperature. The degree of temperature increase or temperature lowering during thermocycling may vary, and a composition may be subjected to thermocycling one or more times as necessary for the present methods.

As used interchangeably herein, the terms “weight equivalents”, “w/w” and “wt/wt” indicate a weight of a second material compared to the weight of a reference amount of a first material. In the present methods, the reference amount may be an amount of starting material. For example, in a reaction with 50 kg of starting material, a solvent employed in the amount of 10.0 weight equivalents would indicate that 500 kg of the solvent was used.

As used interchangeably herein and in the claims, the terms “volume equivalent”, v/v, and “vol/vol” indicate a volume of a second material compared to the weight of a reference amount of a first material. In the present methods, the reference amount may be an amount of starting material. For example, in a reaction with 50 kg of starting material, a solvent employed in the amount of 10.0 volume equivalents would indicate that 500 L of the solvent was used.

As used herein and in the claims, the term “purity” refers to chemical purity of sepiapterin or a form thereof. For example, the amount of sepiapterin present relative to other chemical compounds (e.g., lactoylpterin). In some embodiments, chemical purity may be measured quantitatively, e.g., by HPLC.

### DETAILED DESCRIPTION OF THE INVENTION

### Brief Description of Drawings

FIG. 1 is a schematic representation of the different pathways to reach sepiapterin Form F from sepiapterin crude Form A.

FIG. 2 provides an overlay of X-ray diffraction patterns of reference forms A, D, E, and F.

FIG. 3 provides an overlay of X-ray diffraction patterns of the starting material and solids obtained after each step for the process represented by an embodiment comprising converting crude freebase Form A to 1·HCl salt, freebasing the 1·HCl salt to generate purified free base Form A, thermocycling Form A to generate Form D, and finally drying Form D to produce Form F.

FIG. 4 provides an overlay of X-ray diffraction patterns representing the starting material (free base Form A as input material) and wet solids after thermocycling in a range of acetone/water ratios.

FIG. 5 provides an overlay of X-ray diffraction patterns representing the starting material (free base Form A as input material) and wet solids after thermocycling in a range of DMSO/acetone/water ratios.

FIG. 6 provides an overlay of X-ray diffraction patterns of wet solids obtained after thermocycling of purified sepiapterin free base Form A in various water-rich solvent mixtures.

FIG. 7 provides an overlay of X-ray diffraction patterns of wet solids obtained after thermocycling of unpurified sepiapterin free base Form A in various solvent mixtures.

FIG. 8 provides an overlay of the X-ray diffraction patterns of the sepiapterin free base forms obtained after thermocycling of purified sepiapterin Form A in water/acetone (4:1, v/v) spiked with different levels of lactoylpterin.

FIG. 9 provides an overlay of the X-ray diffraction patterns of the sepiapterin free base forms obtained after thermocycling of purified sepiapterin Form A in DMSO/acetone/water (1:1.67:2.89, v/v/v) spiked with different levels of lactoylpterin.

FIG. 10 provides an overlay of X-ray diffraction patterns of wet cakes obtained after reslurry of sepiapterin Form D wet cake in dry solvents.

FIG. 11 provides an overlay of X-ray diffraction patterns of reference forms D, F, G.

FIG. 12 provides an overlay of X-ray diffraction patterns of Form G obtained from stability experiments without KAPTON® film protection.

FIG. 13 provides an overlay of X-ray diffraction patterns of Form G obtained from stability experiments with KAPTON® film protection.

### PROCESSES FOR PREPARING CRYSTALLINE FORMS OF SEPIAPTERIN FREE BASE

The invention provides methods for preparing crystalline forms of sepiapterin free base (1) (e.g. crystalline Form F of sepiapterin free base). The present disclosure is based, at least in part, on the surprising discovery of a robust, reproducible process for producing large scaleup quantities of highly pure crystalline forms of sepiapterin free base, e.g., crystalline Form F of sepiapterin free base. The disclosure provides methods for reliably and reproducibly prepare crystalline Form F of sepiapterin free base, including multi-gram or multi-kilogram quantities of Form F, a process which required significant development and improvement over previously disclosed processes (e.g., those disclosed in WO 2018/102315 (the '315 publication)).

In some embodiments, the surprising properties of the methods described herein may be attributable to the use of a highly pure sepiapterin free base (e.g., sepiapterin free base with >97% LC purity and less than 1% of lactoylpterin impurity) as a starting material to arrive at the desired form D after thermocycling. To make the process more robust and ensure the desired form D is always obtained post-thermocycling, low temperature neutralization during freebasing was carried out for a prolonged period before slow heat up to 45 °C in the thermocycling stage, thereby ensuring complete neutralization prior to any form change. In some embodiments, these surprising properties may be attributable to the careful control of the time spent carrying out the method steps. In some embodiments, these surprising properties may be attributable to the careful control of the quantity of the reagents. In some embodiments, these surprising properties may be attributable to the careful control of the temperature at which the steps are carried out. In some embodiments, these surprising properties may be attributable to the careful control of the pH. In some embodiments, these surprising properties may be attributable to the careful control of the addition rates of certain reagents. In some embodiments, these surprising properties may be attributable to the careful control of other factors. In some embodiments, the methods described herein are capable of producing highly pure crystalline Form F of sepiapterin free base to the exclusion of other crystalline forms (e.g., crystalline Form D or crystalline Form G). Other surprising and beneficial aspects of the methods of the disclosure are described elsewhere herein.

In another aspect, the disclosure provides a method of preparing a crystalline Form F of sepiapterin free base comprising:
(a) treating sepiapterin free base with an acid to obtain a sepiapterin salt, and freebasing the salt to obtain a purified sepiapterin free base;
(b) thermocycling the purified sepiapterin free base from step (a) in one or more solvents to obtain sepiapterin free base Form D, Form F, or mixtures thereof, and
(c) filtering and drying the sepiapterin free base from step (b) to obtain sepiapterin free base Form F.

In some embodiments, thermocycling of sepiapterin free base in step (b) provides sepiapterin free base Form D. In some embodiments, the method further comprises multiple thermocycling steps to obtain sepiapterin free base Form D. In some embodiments, a second thermocycling step is required if the sepiapterin free base from step (b) is a mixture of sepiapterin free base Form A and Form D or a mixture of sepiapterin free base Form A, Form D, and Form F. In some embodiments, the method further comprises a third thermocycling step to obtain sepiapterin free base Form D. In some embodiments, thermocycling steps are repeated until pure sepiapterin Form D is obtained.

In some embodiments, the sepiapterin free base from step (b) is filtered and resuspended in a solvent prior to the second thermocycling step. In some embodiments, the sepiapterin free base suspended in one or more solvents from step (b) is not filtered prior to the second thermocycling step. In some embodiments, the sepiapterin free base from the second thermocycling step is filtered and resuspended in one or more solvents prior to the third thermocycling step. In some embodiments, the sepiapterin free base suspended in one or more solvents from the second thermocycling step is not filtered prior to the third thermocycling step. In some embodiments, filtration of sepiapterin free base prior to second thermocycling or third thermocycling is optional.

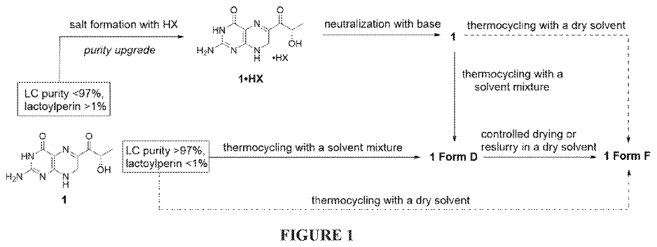

FIG. 1 provides a schematic representation of the steps for the preparation of a substantially pure crystalline F of sepiapterin free base.

Sepiapterin salts for use in the present invention may be any sepiapterin salt including sepiapterin and a proton-donating acid. In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate and sepiapterin sulfate. In some embodiments, the salt of sepiapterin is sepiapterin hydrochloride.

The neutralization step of 1·HCl was found to be non-trivial and, on a number of occasions, the subsequent thermocycling failed to provide the desired form D, and generated undesired Form E instead. Several observations were made that shed light on the mechanism behind this potential failure mode. First, the freebasing step was carried out on a heterogeneous mixture in which both 1·HCl and sepiapterin free base have very low solubility. Second, it was observed that 1·HCl rapidly undergoes disproportionation in acetone/water mixtures to generate free base Form A and HCl before any base is added. Third, subsequent neutralization by addition of a base requires a long age time due to poor mass-transfer. Finally, free HCl is detrimental to the thermocycling and prevents conversion to form D. To make the process more robust and always arrive at the desired form D post-thermocycling, low temperature neutralization during freebasing was carried out for a prolonged period before slow heat up to 45 °C at the thermocycling stage, thereby ensuring complete neutralization prior to any form change of Form A.

In some embodiments, the freebasing in step (a) is done at a temperature less than 25 °C. In some embodiments, freebasing is done at a temperature of from about −2 °C to about 15 °C In some embodiments, the freebasing includes agitating the sepiapterin salt with a base at a temperature of from about −2 °C to about 15 °C In some embodiments, the mixture of sepiapterin salt and the base is agitated for at least 12 h.

In some embodiments, the mixture of sepiapterin salt and the base in step (a) comprises from 9 to 14 weight equivalents (e.g., from 9 to 13, from 9 to 12, from 9 to 11, from 9 to 10, from 10 to 14, from 10 to 13, from 10 to 12, from 10 to 11, from 11 to 14, from 11 to 13, or from 11 to 12 weight equivalents) of water.

In some embodiments, the purified sepiapterin free base in step (a) is sepiapterin Form A. In some embodiments, the sepiapterin free base from step (a) is washed with deionized water prior to step (b).

In some embodiments, the sepiapterin free base in step (b) after thermocycling provides sepiapterin free base Form D, and optionally Form A, Form F, or a mixture thereof. In some embodiments, the sepiapterin free base in step (b) after thermocycling provides a mixture of sepiapterin free base Form A and Form D. In some embodiments, the sepiapterin free base in step (b) after thermocycling provides a mixture of sepiapterin free base Form D and Form F. In some embodiments, the sepiapterin free base in step (b) after thermocycling provides sepiapterin free base Form D.

In some embodiments, the sepiapterin free base in step (b) after thermocycling is an anhydrate. In some embodiments, the sepiapterin free base in step (b) after thermocycling is a hydrate. In some embodiments, the sepiapterin free base in step (b) after thermocycling is a partial hydrate. In some embodiments, the sepiapterin free base in step (b) after thermocycling is a monohydrate. In some embodiments, the sepiapterin free base in step (b) after thermocycling is a dihydrate. In some embodiments, the sepiapterin free base in step (b) after thermocycling is a trihydrate.

In some embodiments, seed crystals of crystalline sepiapterin free base polymorphic form are added in step (b). In some embodiments, the addition of seed crystals is optional. In some embodiments, the seed crystals include crystalline Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, seed crystals of sepiapterin free base Form D, and/or F are added in step (b) to facilitate the production of sepiapterin free base Form D. In some embodiments, the method does not include addition of seed crystals of sepiapterin free base. In a preferred embodiment, seed crystals of crystalline Form F of sepiapterin free base are added in step (b).

In some embodiments, the seed crystals are added in step (b) in an amount of from about 0.005 to about 0.05 weight equivalents. In some embodiments, the seed crystals of crystalline Form F are added in step (b) in an amount of from about 0.005 to about 0.05 weight equivalents.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

Suitable bases may be used in the present invention and include inorganic bases selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, the one or more solvents in step (b) are selected from the group consisting of water, ethanol, acetone, isopropyl alcohol, 2-butanone (methyl ethyl ketone/MEK), tetrahydrofuran (THF), 2-methyltetrahydrofuran (MeTHF), N,N-dimethylacetamide (DMAc), dimethyl sulfoxide (DMSO), N,N-dimethylacetamide (DMF), and mixtures thereof.

In some embodiments, the one or more solvents in step (b) comprise water, acetone, or mixtures thereof. In some embodiments, the one or more solvents in step (b) is a mixture of acetone, water, and DMSO. In some embodiments, the one or more solvents in step (b) comprise acetone and water in a water:acetone vol/vol ratio of from 1:0 to 0:1. In some embodiments, the one or more solvents in step (b) is a mixture of water and acetone in a water:acetone vol/vol ratio of from 4:1 to 1:0.

In some embodiments, the one or more solvents in step (b) is a mixture of water, and MEK in a water:MEK vol/vol ratio of from 1:0 to 0:1. In some embodiments, the one or more solvents in step (b) is a mixture of water and MEK in a water:MEK vol/vol ratio of from 4:1 to 1:0.

In some embodiments, the one or more solvents in step (b) is a mixture of water, and THF in a water:THF vol/vol ratio of from 1:0 to 0:1. In some embodiments, the one or more solvents in step (b) is a mixture of water and THF in a water:THF vol/vol ratio of from 4:1 to 1:0.

In some embodiments, the one or more solvents in step (b) is a mixture of water, and MeTHF in a water:MeTHF vol/vol ratio of from 1:0 to 0:1. In some embodiments, the one or more solvents in step (b) is a mixture of water and MeTHF in a water:MeTHF vol/vol ratio of from 4:1 to 1:0.

In some embodiments, the one or more solvents in step (b) is a mixture of water, and ethanol in a water:ethanol vol/vol ratio of from 1:0 to 0:1. In some embodiments, the one or more solvents in step (b) is a mixture of water and ethanol in a water:ethanol vol/vol ratio of from 4:1 to 1:0.

In some embodiments, the one or more solvents in step (b) is a mixture of water and IPA in a water:IPA vol/vol ratio of from 1:0 to 0:1. In some embodiments, the one or more solvents in step (b) is a mixture of water and IPA in a water:IPA vol/vol ratio of from 4:1 to 1:0.

In some embodiments, the one or more solvents in step (b) is a mixture of water and DMAc in a water:DMAc vol/vol ratio of from 1:0 to 0:1. In some embodiments, the one or more solvents in step (b) is a mixture of water and DMAc in a water:DMAc vol/vol ratio of from 3:1 to 1:0.

In some embodiments, the one or more solvents in step (b) is IPA.

In some embodiments, step (b) comprises the one or more solvents in an amount of at least about 7.5 volume equivalents (solvent volume relative to weight of sepiapterin salt, e.g., mL/g or L/kg). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents.

In some embodiments, the one or more solvents in step (b) is a mixture of water and acetone. In some embodiments, step (b) comprises a mixture of water and acetone in an amount of at least about 7.5 volume equivalents (solvent volume relative to weight of sepiapterin salt). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water and acetone.

In some embodiments, the one or more solvents in step (b) is water. In some embodiments, step (b) comprises at least 7.5 volume equivalents (solvent volume relative to weight of sepiapterin salt) of water. In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volumes therein of water.

In some embodiments, the one or more solvents in step (b) is acetone. In some embodiments, step (b) comprises at least 7.5 volume equivalents (solvent volume relative to weight of sepiapterin salt) of acetone. In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volumes therein of acetone.

In some embodiments, step (b) comprises a mixture of water, acetone, and DMSO in an amount of at least about 7.5 volume equivalents (solvent volume relative to weight of sepiapterin salt). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water, acetone, and DMSO.

In some embodiments, the one or more solvents in step (b) comprise water and DMSO in an amount of at least about 7.5 volume equivalents (solvent volume relative to weight of sepiapterin salt). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water, and DMSO.

In some embodiments, the one or more solvents in step (b) comprise acetone and DMSO in an amount of at least about 7.5 volume equivalents (solvent volume relative to weight of sepiapterin salt). In some embodiments, step (b) about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of acetone, and DMSO.

In some embodiments, thermocycling in step (b) comprises heating purified sepiapterin free base in one or more solvents to a temperature from about 25 °C to about 75 °C followed by cooling to a temperature of less than about 25 °C In some embodiments, the thermocycling step further comprises agitating the purified sepiapterin free base in one or more solvents along with heating and cooling. In some embodiments, thermocycling comprises heating to at least about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling comprises heating from about 40 °C to about 55 °C followed by cooling to a temperature of less than about 25 °C In some embodiments, thermocycling comprises heating from about 40 °C to about 55 °C followed by cooling to a temperature of from about −2 °C to about 15 °C In some embodiments, thermocycling comprises heating to a temperature of at least 35 °C followed by cooling to a temperature of less than 25 °C, about 20 °C, about 15 °C, about 10 °C, about 5 °C, about 0 °C, about −5 °C, about −10 °C, or intermediate temperatures therein. In some embodiments, the thermocycling in step (b) comprises heating the sepiapterin free base in one or more solvents from step (b) to a temperature of from about 30 °C to about 70 °C followed by cooling to a temperature of from about −2 °C to about 15 °C In some embodiments, the thermocycling comprises heating the sepiapterin free base in one or more solvents from step (b) to about 45 °C then cooling to about 5 °C.

In some embodiments, there is an inverse relationship between the heating temperature and heating time. For example, a higher heating temperature may require a shorter heating time, and a lower heating temperature may require a longer heating time to produce crystalline Form D.

In some embodiments, the drying step (c) includes drying at 1 atmosphere pressure. In some embodiments, the drying step (c) includes drying under vacuum (from about −0.09 to about −0.1 MPA). In some embodiments, the drying step (c) includes a stream of nitrogen gas. In some embodiments, step (c) comprises filtering the free base Form D from step (b) and heating the filtered free base to a temperature of from about 20 °C to about 110 °C In some embodiments, the filtered free base in step (c) is dried via heating to a temperature of up to about 100 °C, about 95 °C, about 90 °C, about 85 °C, about 80 °C, about 75 °C, about 70 °C, about 65 °C, about 60 °C, about 55 °C, about 50 °C, about 45 °C, about 40 °C, about 35 °C, about 30 °C, about 25 °C, about 20 °C, or any intermediate temperatures therein. In some embodiments, there is an inverse relationship between the time required for drying and the temperature required for drying. That is, a higher drying temperature may require a shorter drying time, and a lower drying temperature may require a longer drying time to produce crystalline Form F. In some embodiments, the drying is performed at a higher temperature for a shorter time. In some embodiments, the drying is performed at a lower temperature for a longer time. In some embodiments, the drying is monitored by Karl Fisher (KF) titration (oven method) of sepiapterin. In some embodiments, the drying is monitored to prevent the formation of crystalline Form G of sepiapterin free base.

In some embodiments, the drying occurs at a temperature of at least about 20 °C, about 25 °C, about 30 °C, about 35 °C, about 40 °C, about 45 °C, about 50 °C, about 55 °C about 60 °C, about 60 °C, about 70 °C, about 75 °C, about 80 °C, about 85 °C, about 90 °C, about 95 °C, about 100 °C, about 105 °C, about 110 °C, or intermediate temperatures therein. In some embodiments, the drying temperature and time depends upon the water content in the filtered solids. For example, filtered solids with less water content will require a lower drying temperature and shorter time compared to filtered solids with higher water content. In some embodiments, the mixture is filtered under vacuum (from about −0.09 to about −0.1 MPa). In some embodiments, step (d) comprises filtering and drying at 1 atmosphere pressure. In some embodiments, the water content from the filtered mixture depends upon the amounts of solids, agitation speed, and vacuum pressure.

In some embodiments, the drying step (c) comprises drying under vacuum (from about −0.09 to about −0.1 MPa). In some embodiments, the drying step (c) comprises drying under vacuum at a first temperature followed by drying under vacuum at a higher temperature. In some embodiments, the drying step (c) comprises drying under vacuum at about 25 °C followed by heating under vacuum at about 45 °C In some embodiments, the drying step (c) comprises drying under vacuum at about 25 °C for at least about 4 h followed by heating under vacuum at about 45 °C for at least about 4 h. In some embodiments, the drying step (c) comprises drying under vacuum at about 25 °C for at least about 8 h followed by heating under vacuum at about 45 °C for at least about 8 h. In some embodiments, step (d) comprises drying at 1 atmosphere pressure at a temperature of up to about 75 °C.

In some embodiments, steps (a), (b), and (c) are purged/degassed with an inert gas to minimize oxidative degradation of sepiapterin free base or a form thereof. In some embodiments, the inert gas is nitrogen, helium, or argon. In a preferred embodiment, the inert gas is nitrogen.

In some embodiments, the one or more solvents in step (b) are degassed with an inert gas (e.g., degassed by bubbling nitrogen).

In some embodiments, the drying is monitored by Karl Fischer (KF) titration (oven method) of sepiapterin. In some embodiments, the drying is maintained, such that the KF value remains below about 5%. In some embodiments, the drying is maintained, such that the KF value remains below about 4.5%. In some embodiments, the drying is maintained such that the KF value remains at about 4%. In some embodiments, the drying is maintained, such that the KF value remains at about 3.5%. In some embodiments, the drying is maintained such that the KF titration value is maintained at a level to prevent over drying of crystalline Form F of sepiapterin free base to form crystalline Form G of sepiapterin free base.

In some embodiments, sepiapterin free base Form D is washed with acetone prior to step (c). In some embodiments, the acetone is purged with inert gas, e.g., nitrogen, argon, or helium.

In some embodiments, the purity of the sepiapterin free base after drying is a chemical purity (i.e., the purity of sepiapterin to the exclusion of other chemical compounds, such as lactoylpterin). In some embodiments, the chemical purity is determined quantitatively. In some embodiments, the chemical purity is determined by HPLC.

In some embodiments, the sepiapterin free base after drying is qualitatively pure Form F, e.g., as determined by obtaining an X-ray powder diffraction pattern. In some embodiments, the X-ray powder diffraction pattern of the sepiapterin free base after drying does not contain peaks characteristic of other crystalline forms of sepiapterin free base (e.g., of Form A, Form B, Form C, Form D, Form E, and/or Form G). In some embodiments, an X-ray powder diffraction pattern of the sepiapterin free base after drying does not contain peaks characteristic of crystalline Form G. In some embodiments, an X-ray powder diffraction pattern of the sepiapterin free base after drying does not contain peaks characteristic of crystalline Form D.

In some embodiments, the disclosure provides a method of preparing crystalline Form F of sepiapterin free base, which may optionally include additional thermocycling steps to obtain sepiapterin free base Form D. In some embodiments, a second thermocycling step is required if the sepiapterin free base from thermocycling is a mixture of sepiapterin free base Form A and Form D or a mixture of sepiapterin free base Form A, Form D, and Form F. In some embodiments, the method further comprises a third thermocycling step to obtain sepiapterin free base Form D. In some embodiments, thermocycling steps are repeated until pure sepiapterin Form D is obtained.

In another aspect, the disclosure provides a method for preparing crystalline Form F of sepiapterin free base comprising the following steps:
(a) treating sepiapterin free base with an acid to obtain sepiapterin salt, and freebasing the sepiapterin salt to obtain a purified sepiapterin free base;
(b) thermocycling the purified sepiapterin free base from step (a) in one or more solvents to obtain sepiapterin free base Form A, Form D, Form F, or a mixture thereof,
(c) optionally, thermocycling the sepiapterin free base from step (b) in one or more solvents to obtain sepiapterin free base Form D, Form F, or a mixture thereof, and
(d) filtering and drying the sepiapterin free base from step (b) or the optional step (c) to obtain sepiapterin free base Form F.

In some embodiments, the sepiapterin free base from step (b) is filtered and resuspended in a solvent prior to the second thermocycling step. In some embodiments, the sepiapterin free base suspended in one or more solvents from step (b) is not filtered prior to the second thermocycling step.

In some embodiments, filtration of sepiapterin free base prior to second thermocycling or third thermocycling is optional.

In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate, and sepiapterin sulfate. In some embodiments, the salt of sepiapterin is sepiapterin hydrochloride.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

In some embodiments, the base is selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, sepiapterin free base form before thermocycling comprises sepiapterin Form A. In some embodiments, sepiapterin free base form after first or second thermocycling includes sepiapterin Form D, and optionally, Form A and/or Form F.

In some embodiments, the sepiapterin free base form from step (b) is sepiapterin Form D. In some embodiments, the sepiapterin free base form from step (b) is a mixture of sepiapterin Form A and sepiapterin Form D. In some embodiments, the sepiapterin free base is a mixture of sepiapterin Form A, Form D, and Form F. In some embodiments, the sepiapterin free base is a mixture of sepiapterin Form D, and Form F.

In some embodiments, the sepiapterin free base form from step (c) is sepiapterin Form D. In some embodiments, the sepiapterin free base form from step (c) is sepiapterin Form F. In some embodiments, the sepiapterin free base form from step (c) is a mixture of sepiapterin Form D and sepiapterin Form F.

In some embodiments, the sepiapterin free base is filtered in step (b) and resuspended in one or more solvents in step (c). In some embodiments, the sepiapterin free base is not filtered prior to step (c). In some embodiments, the sepiapterin free base in one or more solvents from step (b) is thermocycled directly in step (c) without prior filtration. In some embodiments, the one or more solvents in step (b) and optional step (c) are water and acetone.

In some embodiments, the thermocycling in step (b) and optional step (c) comprises heating purified sepiapterin free base in one or more solvents to a temperature of from about 30 °C to about 75 °C followed by cooling to temperature of less than about 25 °C In some embodiments, thermocycling comprises heating to at least about 25 °C, about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling comprises heating from about 40 °C to about 55 °C, followed by cooling to a temperature of less than about 25 °C In some embodiments, thermocycling comprises heating to a temperature of at least 35 °C. followed by cooling to a temperature of less than about 25 °C, about 20 °C, about 15 °C, about 10 °C, about 5 °C, about 0 °C, about −5 °C, about −10 °C, or intermediate temperatures therein. In some embodiments, the thermocycling in step (b) and optional step (c) comprises heating the sepiapterin free base in one or more solvents from step (b) or optional step (c) to a temperature of from about 30 °C to about 75 °C or higher followed by cooling to a temperature of from about −5 °C to about 15 °C In some embodiments, the thermocycling comprises heating the first or the second sepiapterin free base from step (b) or optional step (c) to about 45 °C then cooling to about 5 °C.

In some embodiments, there is an inverse relationship between the heating temperature and the heating time. For example, a higher heating temperature may require a shorter heating time, and a lower heating temperature may require a longer heating time to produce crystalline Form D.

In some embodiments, step (b) and optional step (c) further comprises adding seed crystals of sepiapterin free base to facilitate crystallization of sepiapterin free base Form D. In some embodiments, the seed crystals include Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, seed crystals of sepiapterin free base Form D, and/or F are added in step (b) and optional step (c) to facilitate the production of sepiapterin free base Form D.

In some embodiments, the one or more solvents in step (b) and optional step (c) are selected from the group consisting of water, ethanol, acetone, isopropyl alcohol, 2-butanone (methyl ethyl ketone/MEK), tetrahydrofuran (THF), 2-methyltetrahydrofuran (MeTHF), N,N-dimethylacetamide (DMAc), dimethyl sulfoxide (DMSO), N,N-dimethylacetamide (DMF), and mixtures thereof.

In some embodiments, the one or more solvents in step (b) and optional step (c) comprise water, acetone, or mixtures thereof. In some embodiments, the one or more solvents in step (b) and optional step (c) is a mixture of acetone, water, and DMSO. In some embodiments, the one or more solvents in step (b) and optional step (c) comprise acetone and water in a water:acetone vol/vol ratio of from 1:0 to 0:1. In some embodiments, the one or more solvents in step (b) is a mixture of water and acetone in a water:acetone vol/vol ratio of 4:1 to 1:0.

In some embodiments, the one or more solvents in step (b) and optional step (c) is a mixture of water and MEK in a water:MEK vol/vol ratio of from 1:0 to 0:1. In some embodiments, the one or more solvents in step (b) and optional step (c) is a mixture of water and MEK in a water:MEK vol/vol ratio of from 4:1 to 1:0.

In some embodiments, the one or more solvents in step (b) and optional step (c) is a mixture of water and THF in a water:THF vol/vol ratio of from 1:0 to 0:1. In some embodiments, the one or more solvents in step (b) and optional step (c) is a mixture of water and THF in a water:THF vol/vol ratio of from 4:1 to 1:0.

In some embodiments, the one or more solvents in step (b) and optional step (c) is a mixture of water and MeTHF in a water:MeTHF vol/vol ratio of from 1:0 to 0:1. In some embodiments, the one or more solvents in step (b) and optional step (c) is a mixture of water and MeTHF in a water:MeTHF vol/vol ratio of from 4:1 to 1:0.

In some embodiments, the one or more solvents in step (b) and optional step (c) is a mixture of water and ethanol in a water:ethanol vol/vol ratio of from 1:0 to 0:1. In some embodiments, the one or more solvents in step (b) and optional step (c) is a mixture of water and ethanol in a water:ethanol vol/vol ratio of from 4:1 to 1:0.

In some embodiments, the one or more solvents in step (b) and optional step (c) is a mixture of water and IPA in a water:IPA vol/vol ratio of from 1:0 to 0:1. In some embodiments, the one or more solvents in step (b) and optional step (c) is a mixture of water and IPA in a water:IPA vol/vol ratio of from 4:1 to 1:0.

In some embodiments, the one or more solvents in step (b) and optional step (c) is a mixture of water and DMAc in a water:DMAc vol/vol ratio of from 1:0 to 0:1. In some embodiments, the one or more solvents in step (b) and optional step (c) is a mixture of water and DMAc in a water:DMAc vol/vol ratio of from 3:1 to 1:0.

In some embodiments, the one or more solvents in step (b) and optional step (c) is IPA.

In some embodiments, step (b) and optional step (c) comprises the one or more solvents in an amount of at least about 7.5 volume equivalents (solvent volume relative to weight of sepiapterin salt, e.g., mL/gm or L/kg). In some embodiments, step (b) and optional step (c) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents.

In some embodiments, the one or more solvents in step (b) and/or step (c) is a mixture of water and acetone. In some embodiments, step (b) and/or step (c) comprises a mixture of water and acetone in an amount of at least about 7.5 volume equivalents (solvent volume relative to weight of sepiapterin salt). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water and acetone.

In some embodiments, sepiapterin free base is washed with acetone prior to step (d). In some embodiments, the deionized water is purged with inert gas, e.g., nitrogen, argon, or helium.

In some embodiments, step (d) comprises filtering the free base from step (b) or optional step (c) and heating the filtered free base under vacuum to a temperature of from about 20 °C to about 75 °C In some embodiments, the filtered free base in step (d) is dried via heating to a temperature of up to about 75 °C, about 70 °C, about 65 °C, about 60 °C, about 55 °C, about 50 °C, about 45 °C, about 40 °C, about 35 °C, about 30 °C, about 25 °C, about 20 °C, or any intermediate temperatures therein.

In some embodiments, there is an inverse relationship between the time required for drying and the temperature required for drying. That is, a higher drying temperature may require a shorter drying time, and a lower drying temperature may require a longer drying time to produce crystalline Form F. In some embodiments, the drying is performed at a higher temperature for a shorter time. In some embodiments, the drying is performed at a lower temperature for a longer time. In some embodiments, the drying is monitored by Karl Fisher (KF) titration. In some embodiments, the drying is monitored to prevent the formation of crystalline Form G of sepiapterin free base.

In some embodiments, the drying occurs at a temperature of at least about 20 °C, about 25 °C, about 30 °C, about 35 °C, about 40 °C, about 45 °C, about 50 °C, about 55 °C, about 60 °C, about 60 °C, about 70 °C, about 75 °C, or intermediate temperatures therein. In some embodiments, the drying temperature and time depend upon the water content in the filtered solids. For example, filtered solids with less water content will require a lower drying temperature and shorter time compared to filtered solids with higher water content. In some embodiments, the mixture is filtered under vacuum. In some embodiments, the water content from the filtered mixture depends upon the amounts of solids, agitation speed, and the vacuum pressure.

In some embodiments, the drying step (d) comprises drying under vacuum (from about −0.09 to about −0.1 MPa). In some embodiments, the drying step (d) comprises drying under vacuum at a first temperature followed by drying under vacuum at a higher temperature. In some embodiments, the drying step (c) comprises drying under vacuum to about 25 °C followed by heating under vacuum to about 45 °C In some embodiments, the drying step (c) comprises drying under vacuum to about 25 °C for at 1 atmosphere pressure followed by heating under vacuum to about 45 °C In some embodiments, step (d) comprises drying at 1 atmosphere pressure at a temperature of up to about 75 °C.

In some embodiments, the entire process is purged/degassed with an inert gas to minimize oxidative degradation of sepiapterin free base or a form thereof. In some embodiments, the inert gas is nitrogen, helium, or argon.

In some embodiments, the one or more solvents in steps (a), (b), and optional step (c) are degassed with an inert gas (e.g., degassed by bubbling nitrogen).

In some embodiments, the drying is monitored by Karl Fischer (KF) titration (oven method) of sepiapterin. In some embodiments, the drying is maintained, such that the KF value remains below about 5.5%. In some embodiments, the drying is maintained such that the KF value remains below about 5%. In some embodiments, the drying is maintained, such that the KF value remains below about 4.5%. In some embodiments, the drying is maintained such that the KF value remains at about 4%. In some embodiments, the drying is maintained, such that the KF value remains at about 3.5%. In some embodiments, the drying is maintained such that the KF titration value is maintained at a level to prevent over-drying of crystalline Form F of sepiapterin free base to form crystalline Form G of sepiapterin free base.

In some embodiments, the purity of the sepiapterin free base after drying is a chemical purity (i.e., the purity of sepiapterin to the exclusion of other chemical compounds, such as lactoylpterin). In some embodiments, the chemical purity is determined quantitatively. In some embodiments, the chemical purity is determined by HPLC.

In some embodiments, the sepiapterin free base after drying is qualitatively pure Form F, e.g., as determined by obtaining an X-ray powder diffraction pattern of the second solid cake. In some embodiments, the X-ray powder diffraction pattern of the sepiapterin free base after drying does not contain peaks characteristic of other crystalline forms of sepiapterin free base (e.g., of Form A, Form B, Form C, Form D, Form E, and/or Form G). In some embodiments, an X-ray powder diffraction pattern of the sepiapterin free base after drying does not contain peaks characteristic of crystalline Form G. In some embodiments, an X-ray powder diffraction pattern of the sepiapterin free base after drying does not contain peaks characteristic of crystalline Form D.

In another aspect, the disclosure provides a method of preparing crystalline Form F of sepiapterin free base, which may include the following steps:
(a) treating sepiapterin free base with an acid to obtain sepiapterin salt, and freebasing the salt to obtain purified sepiapterin free base;
(b) thermocycling the purified sepiapterin free base from step (a) in one or more solvents to obtain sepiapterin free base Form D, Form F, or a mixture thereof,
(c) filtering the sepiapterin free base from step (b) to obtain wet cake comprising sepiapterin free base Form D, Form F, or a mixture thereof, and
(d) drying by reslurrying the wet cake from step (c) in a dry solvent to obtain crystalline sepiapterin free base Form F.

In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate, and sepiapterin sulfate. In some embodiments, the salt of sepiapterin is sepiapterin hydrochloride.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

In some embodiments, the base is selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, freebasing in step (a) is carried out at a temperature of less than 25 °C.

In some embodiments, sepiapterin free base form before thermocycling comprises sepiapterin Form A. In some embodiments, sepiapterin free base form after first or second thermocycling includes sepiapterin Form D, and optionally, Form A and/or Form F. In some embodiments, sepiapterin free base form from step (b) does not include sepiapterin free base Form E in an amount detectable by X-ray powder diffraction.

In some embodiments, thermocycling in step (b) comprises heating purified sepiapterin free base in one or more solvents to a temperature from about 25 °C to about 75 °C followed by cooling to a temperature of less than about 25 °C In some embodiments, thermocycling comprises heating to at least about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling comprises heating from about 40 °C to about 55 °C, followed by cooling to a temperature of less than about 25 °C In some embodiments, thermocycling comprises heating from about 40 °C to about 55 °C, followed by cooling to a temperature of from about −2 °C to about 15 °C In some embodiments, thermocycling comprises heating to a temperature of at least 35 °C followed by cooling to a temperature of less than 25 °C, about 20 °C, about 15 °C, about 10 °C, about 5 °C, about 0 °C, about −5 °C, about −10 °C, or intermediate temperatures therein. In some embodiments, the thermocycling in step (b) comprises heating the sepiapterin free base in one or more solvents from step (b) to a temperature of from about 30 °C and 70 °C followed by cooling to a temperature of from about −2 °C and 15 °C In some embodiments, the thermocycling comprises heating the purified sepiapterin free base in one or more solvents in step (b) to about 45 °C then cooling to about 5 °C.

In some embodiments, there is an inverse relationship between the heating temperature and heating time. For example, a higher heating temperature may require a shorter heating time, and a lower heating temperature may require a longer heating time to produce crystalline Form D and/or Form F.

In some embodiments, step (b) further comprises adding seed crystals of sepiapterin free base to facilitate crystallization of sepiapterin free base Form D. In some embodiments, the seed crystals include Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, seed crystals of sepiapterin free base Form D, and/or F are added in step (b) to facilitate the production of sepiapterin free base Form D. In some embodiments, the method does not include addition of seed crystals of sepiapterin free base.

In some embodiments, the one or more solvents in step (b) are selected from the group consisting of water, ethanol, acetone, isopropyl alcohol, 2-butanone (methyl ethyl ketone/MEK), tetrahydrofuran (THF), 2-methyltetrahydrofuran (MeTHF), N,N-dimethylacetamide (DMAc), dimethyl sulfoxide (DMSO), N,N-dimethylacetamide (DMF), and mixtures thereof.

In some embodiments, the one or more solvents in step (b) comprise water, acetone, or mixtures thereof. In some embodiments, the one or more solvents in step (b) is a mixture of acetone, water, and DMSO. In some embodiments, the one or more solvents in step (b) comprise acetone and water in a water:acetone vol/vol ratio of from 1:0 to 0:1. In some embodiments, the one or more solvents in step (b) is a mixture of water and acetone in a water:acetone vol/vol ratio of 4:1 to 1:0.

In some embodiments, the one or more solvents in step (b) is a mixture of water and MEK in a water:MEK vol/vol ratio of from 1:0 to 0:1. In some embodiments, the one or more solvents in step (b) is a mixture of water and MEK in a water:MEK vol/vol ratio of from 4:1 to 1:0.

In some embodiments, the one or more solvents in step (b) is a mixture of water and THF in a water:THF vol/vol ratio of from 1:0 to 0:1. In some embodiments, the one or more solvents in step (b) is a mixture of water and THF in a water:THF vol/vol ratio of from 4:1 to 1:0.

In some embodiments, the one or more solvents in step (b) is a mixture of water and MeTHF in a water:MeTHF vol/vol ratio of from 1:0 to 0:1. In some embodiments, the one or more solvents in step (b) is a mixture of water and MeTHF in a water:MeTHF vol/vol ratio of from 4:1 to 1:0.

In some embodiments, the one or more solvents in step (b) is a mixture of water and ethanol in a water:ethanol vol/vol ratio of from 1:0 to 0:1. In some embodiments, the one or more solvents in step (b) is a mixture of water and ethanol in a water:ethanol vol/vol ratio of from 4:1 to 1:0.

In some embodiments, the one or more solvents in step (b) is a mixture of water and IPA in a water:IPA vol/vol ratio of from 1:0 to 0:1. In some embodiments, the one or more solvents in step (b) is a mixture of water and IPA in a water:IPA vol/vol ratio of from 4:1 to 1:0.

In some embodiments, the one or more solvents in step (b) is a mixture of water and DMAc in a water:DMAc vol/vol ratio of from 1:0 to 0:1. In some embodiments, the one or more solvents in step (b) is a mixture of water and DMAc in a water:DMAc vol/vol ratio of from 3:1 to 1:0.

In some embodiments, the one or more solvents in step (b) is IPA.

In some embodiments, step (b) comprises the one or more solvents in an amount of at least about 7.5 volume equivalents (solvent volume relative to weight of sepiapterin salt, e.g., mL/g or L/kg)). In some embodiments, step (b) comprises, about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents.

In some embodiments, the one or more solvents in step (b) is a mixture of water and acetone. In some embodiments, step (b) comprises a mixture of water and acetone in an amount of at least about 7.5 volume equivalents (solvent volume relative to weight of sepiapterin salt). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of water and acetone.

In some embodiments, the dry solvent in step (d) is selected from the group consisting of ethanol, acetone, 1-propanol, isopropyl alcohol, acetonitrile (MeCN), 2-butanone (MEK), methyl isobutyl ketone (MIBK), isopropyl acetate (IPAc), toluene, tetrahydrofuran (THF), 2-methyltetrahydrofuran (MeTHF), and mixtures thereof.

In some embodiments, the purity of the sepiapterin free base in step (d) is a chemical purity (i.e., the purity of sepiapterin to the exclusion of other chemical compounds, such as lactoylpterin). In some embodiments, the chemical purity is determined quantitatively. In some embodiments, the chemical purity is determined by HPLC.

In some embodiments, the sepiapterin free base from step (d) is qualitatively pure Form F, e.g., as determined by obtaining an X-ray powder diffraction pattern. In some embodiments, the X-ray powder diffraction pattern of the sepiapterin free base from step (d) does not contain peaks characteristic of other crystalline forms of sepiapterin free base (e.g., of Form A, Form B, Form C, Form D, Form E, and/or Form G). In some embodiments, an X-ray powder diffraction pattern of the sepiapterin free base from step (d) does not contain peaks characteristic of crystalline Form G. In some embodiments, an X-ray powder diffraction pattern of the sepiapterin free base from step (d) does not contain peaks characteristic of crystalline Form D.

In some embodiments, the entire process is purged/degassed with an inert gas to minimize oxidative degradation of sepiapterin free base or a form thereof. In some embodiments, the inert gas is nitrogen, helium, or argon.

In another aspect, the disclosure provides a method of preparing crystalline Form F of sepiapterin free base comprising:
(a) treating sepiapterin free base with an acid to obtain a sepiapterin salt, and freebasing the salt to obtain a purified sepiapterin free base; and
(b) thermocycling the purified sepiapterin free base from step (a) and drying in one or more dry solvents to obtain sepiapterin free base Form F, and optionally Form G.

In some embodiments, the sepiapterin salt is selected from sepiapterin hydrochloride, sepiapterin hydrobromide, sepiapterin oxalate, sepiapterin trifluoroacetate, sepiapterin mesylate, sepiapterin tosylate, sepiapterin besylate, sepiapterin 1,2-ethanedisulfonate, sepiapterin sulfate, sepiapterin phosphate, sepiapterin malonate, sepiapterin L-tartrate, sepiapterin nicotinate, sepiapterin fumarate, sepiapterin gentisate, or sepiapterin glycolate. In some embodiments, the salt of sepiapterin is selected from sepiapterin hydrochloride, sepiapterin mesylate, sepiapterin besylate, and sepiapterin sulfate. In some embodiments, the salt of sepiapterin is sepiapterin hydrochloride. In some embodiments, sepiapterin free base form after thermocycling includes sepiapterin Form D, and optionally, Form A and/or Form F.

In some embodiments, freebasing in step (a) comprises adding a base to sepiapterin salt to adjust the pH. In some embodiments, freebasing comprises adding a base to provide a pH of from about 3 to about 10.5. In some embodiments, freebasing in step (a) comprises adjusting the pH to about 3, about 3.5, about 4, about 4.5, about 5, about 5.5 about 6, about 6.5, about 7, about 7.5, about 8, about 8.5, about 9, about 9.5, about 10, about 10.5, or any intermediate pH values therein. In some embodiments, step (a) comprises adjusting the pH of from about 5 to about 9. In some embodiments, freebasing comprises adjusting the pH of from about 6.5 to about 9. In some embodiments, pH adjustment is done in two stages for a controlled pH increase.

In some embodiments, the base is selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide. In some embodiments, the base is sodium hydroxide.

In some embodiments, freebasing in step (a) is carried out at a temperature of less than 25 °C.

In some embodiments, sepiapterin free base form before thermocycling comprises sepiapterin Form A. In some embodiments, sepiapterin free base form after thermocycling includes sepiapterin Form F, and optionally Form G. In some embodiments, sepiapterin free base form from step (b) does not include sepiapterin free base Form E in an amount detectable by X-ray powder diffraction. In some embodiments, thermocycling in step (b) comprises heating sepiapterin free base in one or more dry solvents to a temperature of from about 25 °C to about 75 °C for at least about 4 h followed by cooling to a temperature of less than about 25 °C for at least about 1 h. In some embodiments, thermocycling comprises heating to at least about 30 °C, about 35 °C, about 40 °C, about 45 °C, about 50 °C, about 55 °C, about 60 °C, about 65 °C, about 70 °C, about 75 °C, or intermediate temperatures therein. In some embodiments, thermocycling step comprises heating to at least about 35 °C for at least about 4 h, about 4.5 h, about 5 h, about 5.5 h, about 6 h, about 6.5 h, about 7 h, about 7.5 h, about 8 h, about 8.5 h, about 9 h or any intermediate periods therein. In some embodiments, thermocycling comprises heating from about 40 °C to about 55 °C, for at least about 6 h followed by cooling to a temperature of less than about 25 °C for at least about 1 h. In some embodiments, thermocycling comprises heating from about 40 °C to about 55 °C, for at least about 6 h followed by cooling to a temperature of from about −2 °C to about 15 °C. for at least about 1 h, In some embodiments, thermocycling comprises heating to a temperature of at least 35 °C followed by cooling to a temperature of less than about 25 °C, about 20 °C, about 15 °C, about 10 °C, about 5 °C, about 0 °C, about −5 °C, about −10 °C, or intermediate temperatures therein. In some embodiments, thermocycling comprises cooling to less than about 25 °C for at least about 1 h, e.g., about 1.5 h, about 2 h, about 2.5 h, about 3 h, about 3.5 h, about 4 h, or any intermediate periods therein. In some embodiments, the thermocycling in step (b) comprises heating the sepiapterin free base in one or more solvents from step (b) to a temperature of from about 30 °C to about 70 °C followed by cooling to a temperature of from about −2 °C to about 15 °C In some embodiments, the thermocycling comprises heating the sepiapterin free base from step (b) to about 45 °C then cooling to about 5 °C.

In some embodiments, there is an inverse relationship between the heating temperature and heating time. For example, a higher heating temperature may require a shorter heating time, and a lower heating temperature may require a longer heating time to produce crystalline Form D.

In some embodiments, step (b) further comprises adding seed crystals of sepiapterin free base to facilitate crystallization of sepiapterin free base Form D. In some embodiments, the seed crystals include Form A of sepiapterin free base. In some embodiments, the seed crystals include crystalline Form B of sepiapterin free base. In some embodiments, the seed crystals include crystalline Form C of sepiapterin free base. In some embodiments, the seed crystals include crystalline Form D of sepiapterin free base. In some embodiments, the seed crystals include crystalline Form E of sepiapterin free base. In some embodiments, the seed crystals include crystalline Form F of sepiapterin free base. In some embodiments, the seed crystals include crystalline Form G of sepiapterin free base. In some embodiments, seed crystals of sepiapterin free base Form D, F and/or G are added in step (b) to facilitate the production of sepiapterin free base Form D. In some embodiments, the method does not include addition of seed crystals of sepiapterin free base.

In some embodiments, the one or more dry solvents in step (b) are selected from the group consisting of acetone, 2-butanone, methyl isobutyl ketone, isopropyl alcohol, dimethyl sulfoxide, N,N-dimethylformamide, N,N-dimethylacetamide, and mixtures thereof.

In some embodiments, the one or more dry solvents in step (b) are isopropyl alcohol, acetone, or a mixture of acetone and DMSO.

In some embodiments, step (b) comprises the one or more dry solvents in an amount of at least about 7.5 volume equivalents (solvent volume relative to weight of sepiapterin salt, e.g., mL/g or L/kg). In some embodiments, step (b) comprises about 7.5 volume equivalents, about 8 volume equivalents, about 9 volume equivalents, about 10 volume equivalents, about 11 volume equivalents, about 12 volume equivalents, about 13 volume equivalents, about 14 volume equivalents, about 15 volume equivalents, about 16 volume equivalents, about 17 volume equivalents, about 18 volume equivalents, about 19 volume equivalents, about 20 volume equivalents, about 25 volume equivalents, about 30 volume equivalents, about 35 volume equivalents, about 40 volume equivalents, about 45 volume equivalents, about 50 volume equivalents, or any intermediate volume equivalents therein of the one or more solvents.

In some embodiments, sepiapterin free base Form F, and optional Form G, from step (b) is filtered and dried at room temperature. In some embodiments, the filtration and drying is done at a temperature of 25 °C or less. In some embodiments, the filtration and drying is done under vacuum (from about −0.09 to about −0.1 MPa) at a temperature of 25 °C or less. In some embodiments, filtration is done without vacuum.

In some embodiments, the sepiapterin free base from step (b) is qualitatively pure Form F, e.g., as determined by obtaining an X-ray powder diffraction pattern. In some embodiments, the X-ray powder diffraction pattern of the sepiapterin free base from step (c) does not contain peaks characteristic of other crystalline forms of sepiapterin free base (e.g., of Form A, Form B, Form C, Form D, Form E, and/or Form G). In some embodiments, an X-ray powder diffraction pattern of the sepiapterin free base from step (d) does not contain peaks characteristic of crystalline Form G. In some embodiments, an X-ray powder diffraction pattern of the sepiapterin free base from step (d) does not contain peaks characteristic of crystalline Form D.

In some embodiments, the entire process is purged/degassed with an inert gas to minimize oxidative degradation of sepiapterin free base or a form thereof. In some embodiments, the inert gas is nitrogen, helium, or argon.

In some embodiments, the crystalline Form D is characterized by an X-ray powder diffraction pattern obtained by irradiation with Cu Kα X-rays having characteristic peaks expressed in degrees 2θ at about 8.9°, about 10.3°, and about 26.0°. In some embodiments, the crystalline Form D is characterized by an X-ray powder diffraction pattern obtained by irradiation with Cu Kα X-rays having peaks expressed in degrees 2θ at about 8.9°, about 10.3°, about 10.9°, about 17.8°, about 24.9°, about 26.0°, about 26.7°, about 26.8°, and about 28.3°.

In some embodiments, the crystalline Form F is characterized by an X-ray powder diffraction pattern obtained by irradiation with Cu Kα X-rays having characteristic peaks expressed in degrees 2θ at about 9.7°, about 10.2°, and about 11.3°. In some embodiments, the crystalline Form F is characterized by an X-ray powder diffraction pattern obtained by irradiation with Cu Kα X-rays having peaks expressed in degrees 2θ at about 9.7°, about 10.2°, about 11.3°, about 14.0°, about 14.6°, about 19.9°, about 22.2°, about 25.3°, and about 32.4°.

### Crystalline Forms of Sepiapterin Free Base

The methods disclosed herein may use solid forms of sepiapterin. In some embodiments, the solid form is an amorphous form. In some embodiments, the solid form is a crystalline polymorph form. In some embodiments, the solid form is a mixture of amorphous and/or crystalline polymorph forms. In some embodiments, the solid form is a salt of sepiapterin.

The polymorphic forms of sepiapterin free base as well as the polymorphic forms of the salts of sepiapterin may be characterized by any suitable method for studying solid state materials. In an embodiment, the polymorphic forms are characterized by XRPD. The XRPD peak positions are expressed as degrees of 2θ. In the X-ray diagram, the angle of diffraction 2θ is plotted on the horizontal axis (x-axis) and the relative peak intensity (background-corrected peak intensity) on the vertical (y-axis). X-ray powder diffraction patterns are measured on, or using instruments comparable to, a PANalytical Empyrean X-ray powder diffractometer with Cu Kα radiation source (Kα1 radiation, wavelength λ=1.54060 Angstrom, Kα2 radiation, wavelength λ=1.544426 Angstrom; Kα2/Kα1 intensity ratio: 0.50). The optical density of the peaks on the film is proportional to the light intensity. The film is scanned using a peak scanner.

As it relates to any of the peaks of X-ray powder diffraction set forth throughout this application, “about” refers to ±0.1, particularly ±0.05, and more particularly ±0.02 of the 2θ values in degrees.

Polymorphic forms of sepiapterin free base and salts are described in WO 2018/102315 and WO 2018/102314, and those that are relevant to this invention are hereby incorporated by reference.

The crystalline Form A of sepiapterin free base is characterized by an X-ray powder diffraction pattern obtained by irradiation with Cu Kα X-rays having peaks expressed in degrees 2θ at about 4.7°, about 7.4°, about 9.5°, about 11.3°, about 15.6°, about 26.2°, and about 27.2°.

The crystalline Form A is characterized by the 2θ peak positions of about 4.7°, about 7.4°, about 9.5°, about 11.3°, about 15.6°, about 26.2°, and about 27.2°.

In an embodiment, the crystalline sepiapterin polymorph Form D is characterized by an X-ray powder diffraction pattern obtained by irradiation with Cu Kα X-rays having characteristic peaks expressed in degrees 2θ at about 8.9°, about 10.3°, and about 26.0°.

In a particular embodiment, the crystalline sepiapterin polymorph Form D is characterized by an X-ray powder diffraction pattern obtained by irradiation with Cu Kα X-rays having peaks expressed in degrees 2θ at about 8.9°, about 10.3°, about 10.9°, about 17.8°, about 24.9°, about 26.0°, about 26.7°, about 26.8°, and about 28.3°.

In an embodiment, the crystalline sepiapterin polymorph Form E is characterized by an X-ray powder diffraction pattern obtained by irradiation with Cu Kα X-rays having characteristic peaks expressed in degrees 2θ at about 6.0°, about 10.6°, and about 12.1°.

In a particular embodiment, the crystalline sepiapterin polymorph Form E is characterized by an X-ray powder diffraction pattern obtained by irradiation with Cu Kα X-rays having peaks expressed in degrees 2θ at about 6.0°, about 10.6°, about 12.10, about 15.9°, about 18.10, about 20.9°, about 22.10, about 24.6°, and about 28.1°.

In an embodiment, the crystalline sepiapterin polymorph Form F is characterized by an X-ray powder diffraction pattern obtained by irradiation with Cu Kα X-rays having characteristic peaks expressed in degrees 2θ at about 9.7°, about 10.2°, and about 11.3°.

In a particular embodiment, the crystalline sepiapterin polymorph Form F is characterized by an X-ray powder diffraction pattern obtained by irradiation with Cu Kα X-rays having peaks expressed in degrees 2θ at about 927 about 10.20, about 11.30, about 14.00, about 14.60, about 19.90, about 22.20, about 25.30, and about 32.40.

In an embodiment, the crystalline sepiapterin polymorph Form G is characterized by an X-ray powder diffraction pattern obtained by irradiation with Cu Kα X-rays having characteristic peaks expressed in degrees 2θ at about 10.00, about 10.60, and about 25.70.

In a particular embodiment, the crystalline sepiapterin polymorph Form G is characterized by an X-ray powder diffraction pattern obtained by irradiation with Cu Kα X-rays having peaks expressed in degrees 2θ at about 10.00, about 10.60, about 11.20, about 15.30, about 15.90, about 22.80, about 24.40, about 25.00, about 25.70, and about 26.60.

In an essentially pure material, crystal Forms A, D, E, F, and G of sepiapterin free base, peaks can be observed at angles of diffraction 2θ as set forth in Table 1.

TABLE 1
XRPD peak positions and intensities for relevant polymorphic forms of sepiapterin

| FORM A | | FORM D | | FORM E | | FORM F | | FORM G | |
|---|---|---|---|---|---|---|---|---|---|
| Position | Relative | Position | Relative | Position | Relative | Position | Relative | Position | Relative |
| [2θ°] | Intensity | [2θ°] | Intensity | [2θ°] | Intensity | [2θ°] | Intensity | [2θ°] | Intensity |
| 4.7 | 47.76 | 8.9 | 100.00 | 6.0 | 100.0 | 9.7 | 98.27 | 5.3 | 8.30 |
| 7.4 | 100.00 | 10.3 | 49.92 | 10.6 | 20.78 | 10.2 | 100.00 | 6.9 | 4.54 |
| 9.5 | 33.54 | 10.9 | 19.96 | 12.1 | 31.95 | 11.3 | 22.47 | 10.0 | 100.00 |
| 11.3 | 19.31 | 11.6 | 2.15 | 15.9 | 12.83 | 14.0 | 5.01 | 10.6 | 69.64 |
| 12.4 | 8.49 | 13.6 | 2.99 | 18.1 | 3.39 | 14.6 | 12.36 | 11.2 | 6.59 |
| 13.4 | 3.60 | 14.2 | 3.45 | 20.9 | 11.63 | 19.9 | 5.63 | 13.5 | 7.52 |
| 14.2 | 8.24 | 14.8 | 2.35 | 22.1 | 2.79 | 21.1 | 3.72 | 15.3 | 26.59 |
| 15.6 | 15.08 | 15.4 | 2.59 | 24.6 | 8.28 | 22.2 | 5.37 | 15.9 | 26.43 |
| 16.4 | 11.97 | 16.4 | 1.55 | 26.1 | 0.88 | 22.7 | 4.04 | 16.0 | 23.41 |
| 17.6 | 8.35 | 17.2 | 2.33 | 28.1 | 7.33 | 24.5 | 2.99 | 16.9 | 4.28 |
| 18.4 | 5.03 | 17.8 | 6.24 | 28.9 | 3.77 | 25.3 | 17.65 | 18.6 | 13.02 |
| 19.8 | 9.18 | 19.6 | 2.62 | 32.1 | 3.57 | 27.2 | 3.10 | 19.3 | 11.90 |
| 21.5 | 5.44 | 20.1 | 2.28 | 37.0 | 1.03 | 32.4 | 5.29 | 20.1 | 7.22 |
| 24.4 | 5.56 | 20.5 | 3.09 | | | 36.7 | 2.72 | 20.8 | 11.01 |
| 26.2 | 35.37 | 20.8 | 2.27 | | | | | 22.8 | 16.77 |
| 27.2 | 19.11 | 21.3 | 3.60 | | | | | 23.5 | 19.60 |
| 28.9 | 5.93 | 22.3 | 4.79 | | | | | 24.4 | 41.45 |
| | | 23.7 | 4.31 | | | | | 25.0 | 23.99 |
| | | 24.9 | 5.19 | | | | | 25.7 | 65.40 |
| | | 26.0 | 41.94 | | | | | 26.6 | 39.64 |
| | | 26.7 | 8.58 | | | | | 27.6 | 13.04 |
| | | 26.8 | 9.17 | | | | | 28.7 | 6.55 |
| | | 27.4 | 3.98 | | | | | 30.8 | 14.76 |
| | | 28.3 | 4.75 | | | | | 32.2 | 9.63 |
| | | 28.7 | 6.60 | | | | | 33.7 | 5.16 |
| | | 29.8 | 3.03 | | | | | | |
| | | 31.8 | 2.72 | | | | | | |
| | | 33.0 | 2.03 | | | | | | |
| | | 35.5 | 1.57 | | | | | | |
| | | 37.1 | 1.09 | | | | | | |

Alternatively, or in addition, crystalline Form A of sepiapterin free base is characterized by a DSC curve showing endothermal peaks at 82.8 °C and 179.8 °C.

Alternatively, the crystalline Form D of sepiapterin free base is characterized by a DSC curve showing endothermal peaks at 42.7 °C, 66.3 °C, and/or 232.9 °C.

Alternatively, the crystalline Form F of sepiapterin free base is characterized by a DSC curve showing endothermal peaks at 71.6 °C and/or 233.4 °C.

In any of the aspects described herein, sepiapterin may exist in multiple tautomeric forms, e.g. either the 1H- or 3H-forms. As an example, sepiapterin free base may exist in either one of the following tautomeric forms:

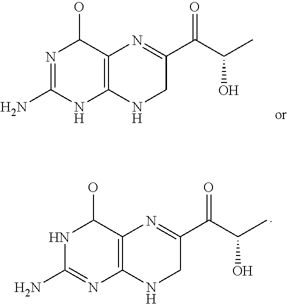

### EXAMPLES

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention. As such, the following examples are provided to teach various aspects of the present invention. These examples represent individual embodiments of the aspects of this invention and one skilled in the art will recognize that additional examples can be generated in order to equally teach the aspects of the present invention.

### Example 1. Preparation of Purified Sepiapterin Hydrochloride from Crude Sepiapterin

The synthetic scheme comprising salt formation of 1 crude with HCl to provide purified 1·HCl is shown below, which is a part of FIG. 1.

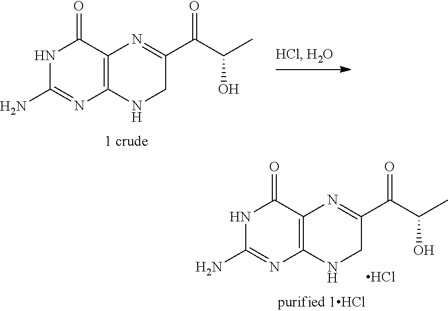 [SMILES: C[C@H](O)C(=O)C1=Nc2c(nc(N)[nH]c2=O)NC1 ; C[C@H](O)C(=O)C1=Nc2c(nc(N)[nH]c2=O)NC1 ; Cl]

### Large-Scale Process

A 10000 L vessel under nitrogen atmosphere was charged with 35% HCl (aq) (1360 kg, 4.71 w/w) and purified water (1346 kg 4.66 w/w), then the contents were cooled to 0 °C and stirred for 1 h. The mixture was then degassed by bubbling nitrogen for 30 minutes. Next, 1 crude (289 kg, material basis for all subsequent steps, 96.6% LC purity, 0.93% lactoylpterin) was charged in portions. Additional degassed and precooled (0 °C.) purified water (580 kg, 2.00 w/w) was used as rinse, after which the mixture was stirred for 1 h. The solids were then isolated by filtration using an agitated filter dryer with the jacket at 0 °C and washed with pre-cooled (0 °C.) and degassed acetone (870 L, 3 L/kg). After deliquoring for 5 h, a wet cake of 1·HCl was obtained (497.56 kg, 98.29% LC purity, 0.15% lactoylpterin).

### Small-Scale Process

A 3 L 4-neck round-bottom flask equipped with overhead stirrer, temperature probe and addition funnel was charged with de-ionized (DI) water (491 mL 4.91 mL/g) and the contents were cooled to 5 °C The flask was purged with nitrogen for around 60 minutes while maintaining the internal temperature at 5 °C Concentrated HCl (472 g, 4.72 w/w) was slowly added and once the addition is complete, the contents were further cooled to 0 °C Next, 1 crude (100 g, assay corrected, 98.28% LC purity, 0.85% lactoylpterin) was added portion-wise while maintaining rigorous inertion of the reactor headspace. To this mixture was added degassed DI water (200 mL, 2.0 mL/g) precooled to 0 °C, after which the mixture was stirred for around 60 minutes. The solids were isolated by filtration and washed with pre-cooled (0 °C.) and degassed acetone (300 mL, 3.0 mL/g). After fully deliquoring, a wet cake of 1·HCl (113.6 g, 99.56% LC purity, 0.15% lactoylpterin) was obtained.

### Example 2. Effect of Acid on Salt Purity

A 100 mL 3-neck round-bottom flask equipped with overhead stirrer, temperature probe and addition funnel was charged with de-ionized (DI) water (24.55 mL 4.91 mL/g) and the contents were cooled to about 5 °C The flask was purged with nitrogen for around 60 minutes while maintaining the internal temperature at about 5 °C Aqueous acid solution was slowly added and once the addition is complete, the contents were cooled to 0 °C Next, 1 crude (5 g, assay corrected, 95.95% LC purity, 1.12% lactoylpterin) was added portion-wise while maintaining rigorous inertion of the reactor headspace. To this mixture was added degassed DI water (10 mL, 2.0 mL/g) precooled to 0 °C, after which the mixture was stirred for around 60 minutes. The solids were filtered, then washed with pre-cooled (0 °C.) and degassed acetone (15 mL, 3.0 mL/g). After being fully deliquored, a wet cake of 1·HX was obtained.

TABLE 2
Formation of 1•HX salts with their respective purities
prepared from 1 having 95.95% LC purity and 1.12% lactoylpterin

| | Equivalents | 1•HX Salt | Lactoylpterin |
|---|---|---|---|
| Acid | Acid Added | Purity (% LC) | (%) |
| Hydrochloric acid | 11 | 98.63 | 0.17 |
| Methanesulfonic acid | 3.7 | 97.29 | 0.45 |
| Sulfuric acid | 1.86 | 97.43 | 0.60 |
| Benzenesulfonic acid | 3.7 | 97.62 | 0.18 |
| p-Toluenesulfonic acid | 3.7 | 96.36 | 1.50 |
| Nicotinic acid | 3.7 | 95.53 | 2.05 |
| Fumaric acid | 1.86 | 92.33 | 1.63 |
| Gentisic acid | 3.7 | 96.54 | 1.51 |
| Glycolic acid | 3.7 | 92.69 | 1.78 |
| Hydrobromic acid | 1.1 | 95.53 | 1.71 |
| 1,2-Ethanedisulfonic acid | 1.1 | 96.22 | 1.31 |
| Oxalic acid | 1.1 | 95.86 | 1.71 |
| Trifluoroacetic acid | 1.1 | 96.22 | 1.31 |

The data in Table 2 demonstrates that hydrochloric acid, methanesulfonic acid, sulfuric acid and benzenesulfonic acid can be used to obtain 1·HX sepiapterin salts with % LC purity of ≥97.0 and lactoylpterin of <1%. For all experiments, the input crude 1 had an LC purity of 95.95% with 1.12% lactoylpterin. XRPD measurements were taken in each case to confirm new diffraction patterns and absence of free form. Each of those salts were then freebased and converted to Form F by thermocycling using the general procedure described in Example 3, with isolated material having the required purity and polymorphic form.

### Example 3. Preparation of Sepiapterin Free Base 1 Form F from 1·HCl

The synthetic scheme comprising freebasing the purified salt 1·HCl and thermocycling to deliver 1 Form F is shown below, which is a part of FIG. 1.

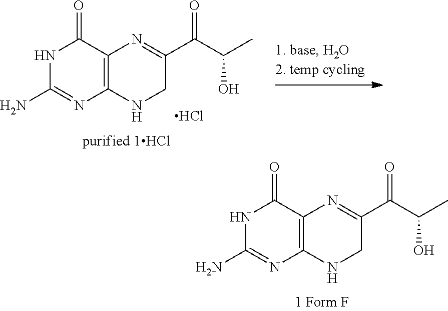 [SMILES: C[C@H](O)C(=O)C1=Nc2c(nc(N)[nH]c2=O)NC1 ; Cl ; C[C@H](O)C(=O)C1=Nc2c(nc(N)[nH]c2=O)NC1]

### Large-Scale Process

A 10000 L vessel was charged with de-ionized (DI) water (3458 kg 12.0 w/w) and the contents were cooled to 5 °C To this solution was added 1 Form F (5.8 kg, 0.02 w/w) as seeds. Next, the 1·HCl (100 g basis of 1 crude) from Example 1 was charged while maintaining the internal temperature at 5 °C Additional degassed DI water (1154 kg, 4.0 w/w), followed by degassed acetone (912 kg, 3.16 w/w) were charged and the internal temperature was maintained at 5 °C The mixture was stirred for 14 h at 5 °C at which time the pH was adjusted to 2.78 by addition of pre-cooled (5 °C.) degassed aqueous sodium hydroxide solution (12 wt %, 472 kg) over 9 h. Subsequently, degassed and precooled (5 °C.) NaOH (1 wt %) solution (29 kg) was charged over 2 h to reach a pH of 6.98. With the pH at an acceptable range of between 6.5 and 7.5, the contents of the flask were warmed to 15 °C and stirred for 3 h. The pH was again checked to ensure it is between 6.5 and 7.5 (measured to be 6.97). The internal temperature was adjusted to 45 °C and the mixture was stirred for 8 h. After this time elapsed, the reaction mixture was cooled to 5 °C and stirred for an additional 3 h. A sample of the solids was checked by XRPD, and it was confirmed to be Form D. The slurry was then transferred to an agitated filter dryer and deliquored. To the isolated solids was added a degassed mixture of water (2642 kg, 9.06 w/w) and acetone (659 L, 2.27 L/kg) and the resulting slurry was stirred at 45 °C for about 8 h within the filter dryer. The contents were next cooled to 5 °C and stirred for 3 h followed by deliquoring. The solids were washed with precooled (5 °C.) and degassed acetone (1450 L, 5.02 L/kg), deliquored, and then washed again with precooled (5 °C.) and degassed acetone (1451 L, 5.02 L/kg). The jacket temperature was adjusted to 25 °C and dried under vacuum (from about −0.095 to about −0.099 mPA) for 13 h at which point the jacket temperature was increased to 45 °C and dried further under vacuum (from about −0.097 to about −0.100 mPa) for 21 h. The solids were confirmed to be Form F by XRPD and then sieved with a 10-20 mesh screen before packaging to provide 1 Form F (252 kg, 87% yield from 1 crude).

### Small-Scale Process

A 5 L 4-neck round-bottom flask equipped with overhead stirrer, reflux condenser, temperature probe and addition funnel was charged with de-ionized (DI) water (1200 mL 12.0 mL/g) and the contents were cooled to 5 °C The vessel was purged with nitrogen for about 60 minutes while maintaining the internal temperature at 5 °C To this solution was added 1 Form F (2 g, 0.02 w/w) as seeds. Next, the solid 1·HCl (100 g basis of 1 crude) from Example 1 was charged while maintaining strict exclusion of air. Additional degassed DI water (400 mL, 4.0 mL/g), followed by degassed acetone (400 mL, 4.0 mL/g) were charged and the internal temperature was adjusted to 5 °C The mixture was stirred for about 14 h at 5 °C at which time the pH was adjusted to 6.73 with pre-cooled (5 °C.) and degassed aqueous sodium hydroxide solution (3 wt %). With the pH at an acceptable range of between 6.5 and 7.4, the contents of the flask were warmed to 15 °C and stirred for about 3 h. The pH was again checked to ensure it was still between 6.5 and 7.4 (measured to be 7.06). The internal temperature was adjusted to 45 °C and stirred for about 8 h. After this time elapsed, the reaction mixture was cooled to 5 °C and stirred for an additional 3 h. A sample of the solids was checked for form by XRPD, and it was confirmed as 1 Form D. The solids were then isolated by filtration and deliquored to provide an intermediate wet cake.

A clean and dry 2 L 4-neck round-bottom flask equipped with overhead stirrer, reflux condenser, temperature probe and addition funnel was charged with DI water (1294 mL, 12.94 mL/g) and the contents were adjusted to 20 °C The vessel was purged with nitrogen for about 60 min while maintaining the internal temperature at 20 °C To this solution was added degassed acetone (323 mL, 3.23 mL/g) and the contents were transferred to the intermediate wet cake contained within an inerted filter vessel. The solid-liquid mixture was heated to 45 °C and then stirred for about 8 h. The mixture was next stirred for about 3 h at 5 °C at which time the solids were filtered and deliquored. The solids were then washed with precooled (5 °C.) and degassed acetone (100 mL, 1.0 mL/g).

Next, the solids were charged with precooled (5 °C.) and degassed acetone (500 mL, 5.0 mL/g). The resultant slurry was then stirred for about 3 h at 5 °C after which time the solids were filtered and deliquored. To the solids were again charged precooled (5 °C.) and degassed acetone (500 mL, 5.0 mL/g). The mixture was then stirred for about 3 h at 5 °C after which time the solids were filtered and deliquored. The solids were next dried at 25 °C under vacuum (from about −0.095 to about −0.099 MPa) for about 12 h. The temperature was then increased to 40 °C and the solids were dried further under vacuum (from about −0.097 to about −0.1 MPa) for 24 h. The polymorphic form of the dried solids was checked by XRPD and confirmed to be 1 Form F (70 g, 70% isolated yield from crude 1).

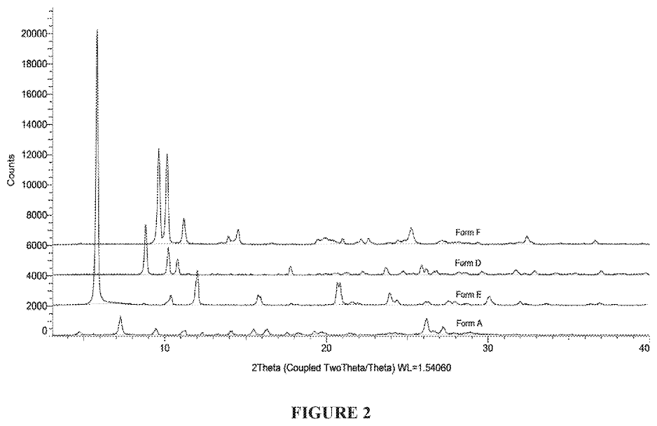

FIG. 2 provides an overlay of X-ray diffraction diagrams of reference forms A, D, E, and F.

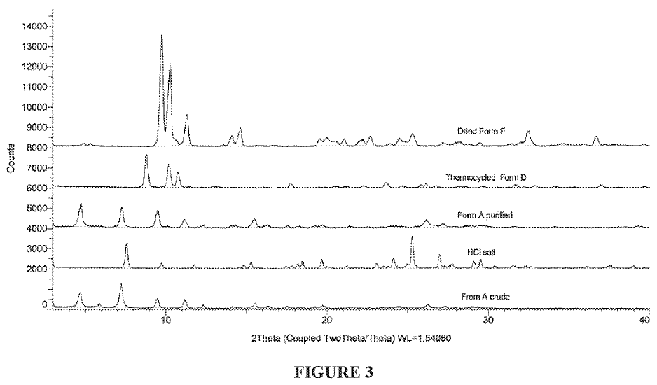

FIG. 3 provides an overlay of X-ray diffraction diagrams of the starting material and solids obtained after each step of the process.

### Example 4. Effect of Base on the Salt-Break of 1·HCl

A 100 mL 3-neck round-bottom flask equipped with magnetic stir bar, reflux condenser, temperature probe, calibrated pH probe (Mettler LE438) and addition funnel was charged with de-ionized (DI) water (60 mL 12.0 mL/g) and the contents were cooled to 5 °C The vessel was purged with nitrogen for about 60 minutes while maintaining the internal temperature at 5 °C To this solution was added 1 Form F (0.1 g, 0.02 w/w) as seeds. Next, the solid 1·HCl (5 g basis of 1 crude) from Example 1 was charged while maintaining strict exclusion of air. Additional degassed DI water (20 mL, 4.0 mL/g), followed by degassed acetone (20 mL, 4.0 mL/g) were charged and the internal temperature was adjusted to 5 °C The mixture was stirred at 5 °C for about 14 h after which time the pH was adjusted to ~6.73 using pre-cooled (5 °C.) and degassed aqueous base solution (12% w/w for NaOH, 6% w/w for all other bases, prepared ahead of time) in seven portions with the pH recorded after each addition. With the pH at an acceptable range (between 6.5 and 7.4), the contents of the flask were warmed to 15 °C and stirred for about 3 h. The pH was again checked to ensure it was still between 6.5 and 7.4. The internal temperature was adjusted to 45 °C and the mixture was stirred for about 8 h. After this time elapsed, the reaction mixture was cooled to 5 °C and stirred for about 3 h. A sample of the solids was checked for form by XRPD, which confirmed they were Form D. The solids were then filtered and dried using the procedure provided in Example 3 to obtain product 1 Form F.

TABLE 3
Effect of Base on conversion of 1•HCl to 1 Form F

| | Equivalents required to | Dry cake | % LC Purity of |
|---|---|---|---|
| Base | reach a pH of 6.7 | Form | Isolated Solids |
| NaOH | 0.97 | F | 98.53 |
| KOH | 1.31 | F | 98.43 |
| K₂CO₃ | 0.77 | F | 98.34 |
| K₃PO₄ | 0.79 | F | 98.29 |
| NaHCO₃ | 1.87 | F | 98.22 |
| K₂HPO₄ | 2.05 | F | 98.27 |

The data from Table 3 shows that NaOH, KOH, K₂CO₃, K₃PO₄, NaHCO₃, and K₂HPO₄ can be used interchangeably as they are all effective in the salt break/freebasing to provide a dry cake comprising 1 Form F with acceptable LC purity of ≥97%.

### Example 5. Effect of pH and Temperature on Freebasing of 1·HCl

The neutralization/freebasing step of 1·HCl was found to be non-trivial and, on a number of occasions, the subsequent thermocycling failed to produce the desired Form D, and instead generated undesired Form E. Several observations were made that shed light on the mechanism behind this potential failure mode.

Just as is the case for the salt formation step (Example 1), the salt break/freebasing step (Example 3) was carried out on a heterogeneous mixture without achieving full dissolution. When following low temperature (e.g., 0 °C.) neutralization with online pH monitoring, large fluctuations in apparent pH were observed, which eventually stabilized over a period of time, and were found to be sensitive to the amount of solvent present. For example, at 0 °C neutralization temperature, neutralization time was 30 mins for a slurry density of 23 mg/mL, but 150 minutes for a slurry density of 33 mg/mL.

Further, if the mixture was thermocycled/heated to 45 °C before complete pH neutralization (as judged by stability of the pH trace), undesired Form E was obtained, which could not be converted to Form F with additional thermocycling. It was surprisingly observed that 1·HCl, when mixed with water/acetone solvent mixtures in absence of any base, underwent spontaneous disproportionation to freebase 1 Form A and HCl. Based on these combined observations, it was concluded that free HCl released quickly when subjected to mixing with solvent mixtures in absence of any base, but was trapped within the crystal lattice, thereby only reacting slowly with the base solution. Because of these mass-transfer limitations, base neutralization was deemed non-trivial and adequate neutralization time at low temperatures was critical before advancing to the heat-up stage.

Table 4 summarizes experiments conducted at different temperatures and pH values to evaluate their impact on the initial pre-thermocycled polymorph after pH adjustment and aging, as well as the post-thermocycled polymorph. The polymorphic form after an aging time of 18 hours was monitored by Raman probe which discriminates Form A (1640 cm⁻¹) from Form D (1660 cm⁻¹) while the polymorphic form of final isolated solids was determined by XRPD analysis.

TABLE 4
Impact of temperature and pH on the salt-
break of 1•HCl after an 18 h hold time

| | Hold | Polymorphic | Polymorphic |
|---|---|---|---|
| | Temp | Form after pH adjustment | Form after Thermocycling |
| pH | (°C) | and 18 h hold time (Raman) | at 45 °C (XRPD) |
| 1.5 | 0 | A | D + F |
| 1.5 | 10 | A | D + F |
| 1.5 | 20 | A + D | E (undesired) |
| 1.5 | 30 | A + D | E (undesired) |
| 1.5 | 45 | A + D | E (undesired) |
| 3.0 | 0 | A | D + F |
| 3.0 | 10 | A | D + F |
| 3.0 | 20 | A + D | Unknown Form Mix |
| 3.0 | 20 | A + D | Unknown Form Mix |
| 3.0 | 30 | A + D | Unknown Form Mix |
| 3.0 | 30 | A + D | Unknown Form Mix |
| 3.0 | 45 | A + D | D + F |
| 7.0 | 0 | A | D + F |
| 7.0 | 10 | A | D + F |
| 7.0 | 20 | A + D | D + F |
| 7.0 | 30 | A + D | D + F |
| 7.0 | 45 | A + D | D + F |
| 9.0 | 0 | A | D + F |
| 9.0 | 10 | A | D + F |
| 9.0 | 20 | A + D | D + F |
| 9.0 | 30 | A + D | D + F |
| 9.0 | 45 | A + D | D + F |

The XRPD data from Table 4 was reformatted and presented in Table 5 in a matrix format below for easier viewing.

TABLE 5
Impact of temperature and pH during salt-break on
sepiapterin free base obtained after thermocycling

pH

| Temperature (°C) | 1.5 | 3 | 7 | 9 |
|---|---|---|---|---|
| 45 | E | D + F | D + F | D + F |
| 30 | E | Mixture | D + F | D + F |
| 20 | E | Mixture | D + F | D + F |
| 10 | D + F | D + F | D + F | D + F |
| 0 | D + F | D + F | D + F | D + F |

The data from Table 5 shows that undesired 1 Form E was obtained when the process slurry was held at pH 1.5 at temperatures of 20 °C, 30 °C, and 45 °C or a mixture including undesired forms when held at pH of 3.0 at temperatures of 20 °C or 30 °C The data further shows that a mixture of form D and F was consistently obtained when the process slurry was held at pH 7 or above at all tested temperatures. A potential failure mode that was made clear was the combination of undercharging base with higher temperature holds. Furthermore, it was found that very fast pH adjustment could also result in undesired Form E. These data taken were then utilized to design a salt break/freebasing process where base was slowly added at a low temp (~5 °C.), followed by temperature increase to 15 °C and an age time of several hours to ensure complete neutralization before heat-up to 45 °C in order to achieve form conversion, then re-cooling to 0-15 °C to minimize liquor losses.

### Example 6. Thermocycling in DMSO/Acetone/Water (1:1.67:2.89, v/v/v) Mixture for Converting Purified 1 Form A to 1 Form D

A 500 mL cylindrical vessel equipped with an overhead stirrer, temperature probe and an addition funnel was charged was charged with DMSO (90 mL, 4.5 mL/g) and water (10 mL, 0.5 mL/g). The solvents were purged with nitrogen to remove dissolved oxygen. The internal temperature was adjusted to 25 °C and purified 1 Form A (20.0 g, assay corrected) was charged followed by the addition of a mixture of degassed acetone and DI water (30 mL, 1:1 acetone/DI water, v/v, 1.5 mL/g) to provide a homogenous solution. While maintaining rigorous exclusion of air, seeds of 1 Form F (0.5 g, 0.5% w/w) were charged followed by an additional 10 mL degassed solution of acetone/DI water (1:1 v/v, 1 mL/g) and the resultant mixture was stirred for 1 h at 25 °C. To the developed seedbed was slowly added a solution of degassed acetone/DI water (260 mL, 1:1 acetone/water, v/v, 13 mL/g,) over a period of 4 h. The mixture was then aged for 12 h at 25 °C after which time, degassed DI water (100 mL, 5 mL/g) was charged over a period of 1 h. Next, the reaction mixture was cooled to 5 °C over a period of 2 h and then aged for an additional 15 h. The resulting solids were filtered and washed with water three times (80 mL, 4 mL/g for each wash). The solids were dried at 25 °C under vacuum (from about −0.095 to about −0.099 MPa) for at least 12 h, followed by drying under vacuum (from about −0.097 to about −0.1 MPa) for further 12 h at a temperature of about 40 °C to obtain sepiapterin free base. The XRPD of sepiapterin free base matched with 1 Form F (18 g, 90% yield).

### Example 7. Effect of DMSO/Acetone/Water Ratios on Thermocycling

Process limits with respect to solvent composition were determined with varying ratios of acetone, water, and DMSO. A 100 mL cylindrical vessel equipped with a magnetic stir-bar and temperature probe was charged with purified 1 Form A (2.0 g, assay corrected, 98.75% LC purity, 0.23% lactoylpterin) followed by a degassed solvent comprising water, acetone, DMSO or mixtures thereof as indicated in Table 6. Seeds of 1 Form F (10 wt % used to maximize turnover kinetics) were added. The internal temperature of the vessel was set adjusted to 35 °C and the contents were aged while mixing for 5 h. The reaction mixture was then cooled to 25 °C over a period of 2-5 h followed by additional aging for 2-5 h. A sample of the solids (wet form) was checked for form by XRPD. The solids were then filtered and washed with 2 mL/g of a solvent mixture equal in composition to the crystallization solvent mixture. The solids were subsequently dried at 40 °C under vacuum (from about −0.09 to about −0.1 MPa) for at least about 16 h. The solids were checked for form by XRPD to ensure drying to Form F was complete.

TABLE 6
Outcome of thermocycling for different DMSO/acetone/water ratios

| | | | | | LC | |
|---|---|---|---|---|---|---|
| | Solvent | Temp | Polymorphic Form | | Purity | Yield |
| Solvent System | Volume Ratio | (°C) | Wet cake | Dry cake | (%) | (%) |
| acetone/water | 0:1 | 50 | D | F | 98.47 | 87 |
| (0 v/15 v) | | | | | | |
| acetone/water | 0.33:0.66 | 50 | D | F | 98.53 | 86 |
| (5 v/10 v) | | | | | | |
| acetone/water | 0.5:0.5 | 50 | D + Trace F | F | 98.60 | 80 |
| (7.5 v/7.5 v) | | | | | | |
| acetone/water | 0.66:0.33 | 50 | D + F | F | 98.68 | 87 |
| (10 v/5 v) | | | | | | |
| acetone/water | 0.938:0.062 | 50 | F + Trace D | F | 98.40 | 95 |
| (15 v/1 v) | | | | | | |
| acetone/water | 1:0 | 50 | F | F | 98.05 | 95 |
| (15 v/0 v) | | | | | | |
| acetone/water/DMSO | 0.33:0.33:0.33 | 35 | F + Trace D | F | 98.35 | 57 |
| (7.5 v/7.5 v/7.5 v) | | | | | | |
| acetone/water/DMSO | 0.66:0:0.33 | 35 | F | F | 98.35 | 81 |
| (10 v/0 v/5 v) | | | | | | |
| acetone/water/DMSO | 0.29:0.29:0.42 | 35 | D | F | 98.36 | 4 |
| (10 v/10 v/15 v) | | | | | | |
| acetone/water/DMSO | 0:0.66:0.33 | 35 | D | ND | 98.43 | 90 |
| (0 v/10 v/5 v) | | | | | | |

Table 6 provides data showing conversion of purified 1 Form A to 1 Form D by thermocycling at 50 °C in acetone/water mixtures and at 35 °C in acetone/DMSO/water mixtures, followed by cooling to 25 °C and drying at 40 °C From the data, it is evident that acetone and water could be used in almost any ratio while obtaining relatively high isolated yield and purity of 1 Form F after drying. There were higher mother liquor losses when both solvents were present at equal amounts indicating a synergistic solvent effect on solubility. When using DMSO, the solubility was high enough that an initial solution could be obtained (as is the case in Example 6) and supersaturation achieved by adding water and/or acetone. The relative percentage of DMSO is ideally kept low to maximize yield without affecting purity.

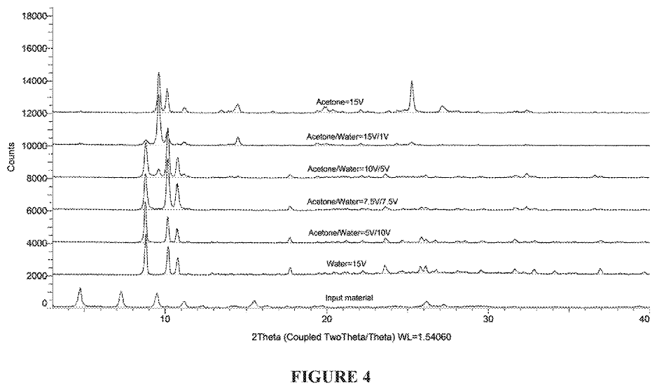

FIG. 4 provides an overlay of X-ray diffraction patterns representing the starting material (freebase Form A as input material) and wet solids after thermocycling in a range of acetone/water ratios.

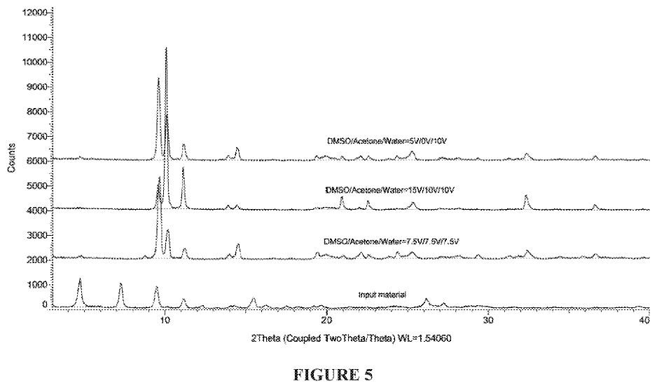

FIG. 5 provides an overlay of X-ray diffraction patterns representing the starting material (freebase Form A as input material) and wet solids after thermocycling in a range of DMSO/acetone/water ratios.

### Example 8. Thermocycling with High-Purity 1 Form A in Different Solvent Mixtures

A 100 mL cylindrical vessel equipped with a magnetic stir-bar and temperature probe was charged with crude 1 Form A (2.0 g, assay corrected, 98.57 LC Purity, 0.23% lactoylpterin) followed by a degassed solvent mixture (36 mL, 18 mL/g) as indicated in Table 7. High seed loadings of 1 Form F (10 wt %) were added to maximize turnover kinetics, then the reaction vessel was purged with nitrogen. The internal temperature was set to 50 °C and the contents were then aged while mixing for 5 h. The reaction mixture was then cooled to 25 °C over a period of 2-5 h followed by additional aging for 2-5 h. A sample of the solids (wet form) were checked for form by XRPD. The resulting solids were filtered and washed with 2 mL/g of a solvent mixture equal in composition to the crystallization solvent mixture. The solids were dried at 40 °C under vacuum (from about −0.09 to about −0.1 MPa) for at least 16 h. The solids were checked for form by XRPD to ensure drying to Form F was complete.

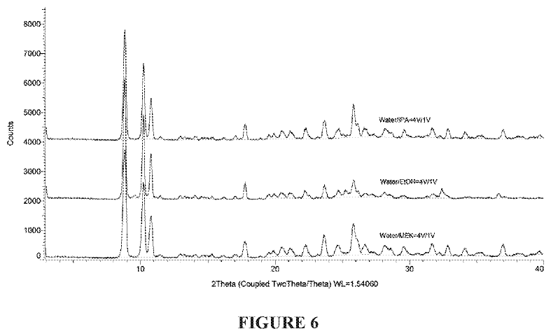

FIG. 6 provides an overlay of X-ray diffraction diagrams of wet solids obtained after thermocycling of purified sepiapterin free base in various water rich solvent mixtures.

TABLE 7
Thermocycling in various solvent/water
mixtures using high purity 1 Form A

| Solvent System | Temperature | Polymorphic form | % LC Purity |
|---|---|---|---|
| (v/v) | (°C) | in wet cake | After Isolation |
| 4:1 water/acetone | 45 | D + F | 99.63 |
| 4:1 water/MEK | 45 | D | 99.44 |
| 4:1 water/THF | 45 | D + F | 98.94 |
| 4:1 water/MeTHF | 45 | D | 99.17 |
| 4:1 water/EtOH | 45 | D | 98.99 |
| 4:1 water/IPA | 45 | D | 99.05 |
| 3:1 water/DMAc | 50 | D | 98.69 |
| IPA | 55 | F | 93.48 |

Table 7 provides experimental results of thermocycling with purified 1 Form A in various water-rich cosolvent mixtures. It was found that acetone, MEK, THF, 2-MeTHF, EtOH, IPA and DMAc served as good cosolvents. It was further observed that thermocycling in dry IPA directly provided 1 Form F without any sign of Form D in the XRPD pattern. This was a unique finding and represents one of the only pure solvents capable of this direct transformation.

### Example 9. Thermocycling Experiments with Crude 1 Form A in Different Solvent Mixtures

Without carefully controlling the purity of 1 Form A, form turnover via thermocycling is capricious and prone to failure. As such, we repeated the thermocycling on crude 1 Form A in several solvent mixtures that support form turnover with purified 1 Form A. Experiments were run using the same procedure provided in Example 8 except that crude 1 was used, having 96.80 LC % purity with 0.97% lactoylpterin.

TABLE 8
Thermocycling crude 1 Form A in various solvent/water mixtures

| | Temperature | Polymorphic form | % LC |
|---|---|---|---|
| Solvent System | (°C) | in wet cake | Purity |
| acetone/water | 50 | A + E | 96.10 |
| (0 v/15 v) | | | |
| acetone/water | 50 | A + E | 97.07 |
| (7.5 v/7.5 v) | | | |
| acetone/water | 50 | A + E | 95.51 |
| (15 v/1 v) | | | |
| acetone/water/DMSO | 35 | E | 87.50 |
| (7.5 v/7.5 v/7.5 v) | | | |
| acetone/water/DMSO | 35 | E | 93.74 |
| (10 v/0 v/5 v) | | | |
| IPA | 55 | F | 95.38 |

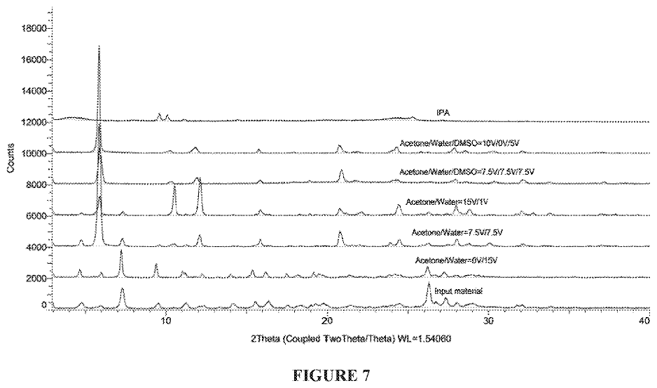

FIG. 7 provides an overlay of X-ray diffraction patterns of wet solids obtained after thermocycling of unpurified sepiapterin free base Form A in various solvent mixtures. The data from Table 8/FIG. 7 shows that crystallization of crude 1 Form A in varying solvent ratios of acetone, water, and DMSO fail to provide the desired 1 Form F. Neither multiple rounds of thermocycling nor higher temperatures were effective in enriching the desired form. We attribute these outcomes to specific and potent inhibition of Form F crystallization by the structurally similar lactoylpterin. However, it was surprisingly observed that thermocycling of crude 1 Form A in IPA at 55 °C did uniquely lead to the conversion to Form F, albeit with low crystallinity. IPA was the only solvent found to be capable of promoting this slurry form turnover with low-purity 1 Form A.

Lactoylpterin Spiking into Pure 1 Form A

While overall purity plays a role, it became clear that the level of lactoylpterin was particularly correlated with thermocycling outcomes. In order to support this hypothesis, experiments were conducted using the procedures outlined in Example 8, taking purified 1 Form A and spiking with varying levels of lactoylpterin. The amounts of lactoylpterin listed in Table 9 correspond to measured % LC area after spiking. These experiments were performed both with the water/acetone (4:1, v/v) and DMSO/acetone/water (1:1.67:2.89, v/v/v) solvent mixtures.

TABLE 9
Impact of lactoylpterin level on the conversion
of purified 1 Form A to 1 Form F

| | | Polymorphic Form of 1 after |
|---|---|---|
| Solvent System | % Lactoylpterin | Thermocycling |
| water/acetone (4:1) | 0.26 | D + F |
| | 0.5 | D + F |
| | 1.0 | D + F |
| | 1.1 | A + D + E |
| | 1.3 | A + D + E |
| | 1.5 | D + E |
| DMSO/acetone/water | 0.35 | D + F |
| (1:1.67:2.89) | 0.54 | D + F |
| | 0.89 | A + trace E |
| | 1.13 | E + trace A |
| | 1.63 | E |

As indicated in Table 9, thermocycling of purified 1 Form A with higher levels of lactoylpterin, lead to unconverted Form A and/or the formation of undesired Form E. In a water/acetone (4:1 v/v) solvent system, the critical level of lactoylpterin was found to lie between 1.0 and 1.1 LC area %. The critical level of lactoylpterin for a DMSO/acetone/water system (1:1.67:2.89 v/v/v) was even lower at between 0.54 and 0.89 LC area %. These results demonstrate that the amount of lactoylpterin should always be kept below 1%, and preferably lower, to avoid formation of undesired Form E. Processes that produce Form E are considered unsuitable as there is no reliable method to convert it to Form D or F.

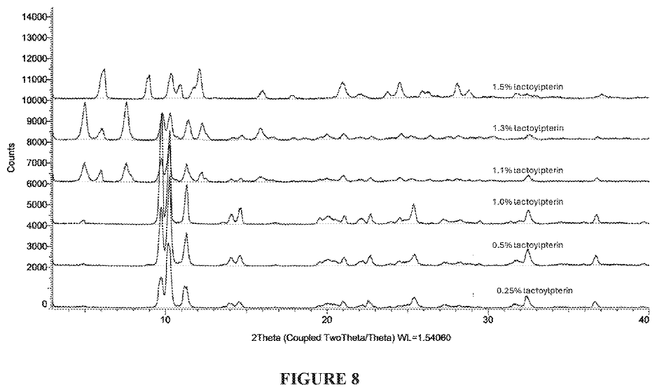

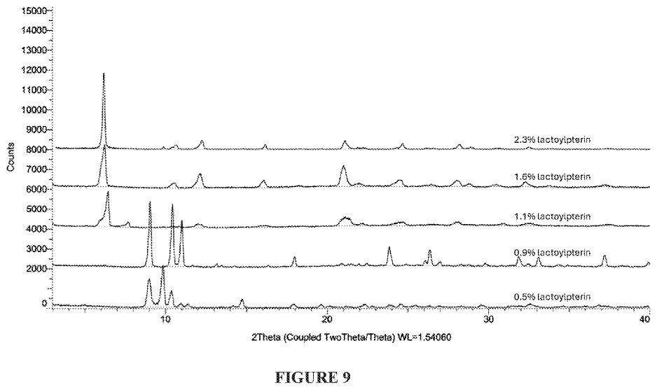

FIG. 8 provides an overlay of the X-ray diffraction patterns of the sepiapterin free base forms obtained after thermocycling of purified sepiapterin Form A in water/acetone (4:1, v/v) spiked with different levels of lactoylpterin. FIG. 9 provides an overlay of the X-ray diffraction patterns of the sepiapterin free base forms obtained after thermocycling of purified 1 Form A in DMSO/acetone/water (1:1.67:2.89, v/v/v) spiked with different levels of lactoylpterin.

### Example 10. Direct Conversion of Sepiapterin Freebase Form A to Form F by Vacuum Drying

This example provides an alternative method for generating sepiapterin freebase Form F directly from Form A, while avoiding thermocycling. In principle this represents a more direct route to Form F, but we have found it to be unreliable, often failing to provide qualitatively pure Form F.

The outcomes of nine drying experiments at different scales with a range of input purities are presented in Table 10. Input materials, either crude or purified sepiapterin freebase Form A containing 10-15 wt % water, were dried under vacuum (from about −0.09 to about −0.1 MPa) for a period of 24 h at the indicated temperature. The first two experiments, including one at manufacturing scale, show successful form conversions that were accomplished with a modest temperature of 45 °C However, all other attempts to affect this polymorph conversion by drying different batches failed. Based on experiments where more pure material did not convert from Form A, we infer that purity (at least in the window of 97% and above) is not a critical factor. Two experiments were also performed comparing relatively strong (−0.1 MPa) and somewhat weaker vacuum (−0.07 MPa) but no difference was found (Experiments 8, 9). Based upon the data, the dominant factors remain unknown but are likely to be related to one or more of the following: scale/equipment setup, particle size, crystal surface activation, mixing effects, temperature, and vacuum.

TABLE 10
Reproducibility in drying sepiapterin freebase Form A to produce Form F

| | | | 1 Form A | | | Final Form | Water |
|---|---|---|---|---|---|---|---|
| | | | (% LC | Lactoylpterin | Drying | (End of | Content |
| Experiment | Scale | | Purity) | (%) | Temp/time | Experiment) | (%) |
| 1 | 180 | g | 98.04 | 0.84 | 45 °C/24 h | F | 4.2 |
| 2 | 136.2 | kg | 97.4 | 1.0 | 45 °C/24 h | F + trace A | 3.7 |
| 3 | 10 | g | 97.95 | 1.0 | 45 °C/24 h | A | 6.1 |
| 4 | 10 | g | 96.58 | 1.88 | 45 °C/24 h | A | 5.88 |
| 5 | 0.3 | g | 99.02 | 0.49 | 45 °C/24 h | A | 0.02 |
| 6 | 0.3 | g | 97.08 | 1.63 | 45 °C/24 h | amorphous | 0.02 |
| 7 | 5 | g | 98.09 | 0.69 | 40 °C/24 h | A + F | 6.24 |
| 8 | 20 | g | 98.54 | 0.79 | 45 °C/24 h/−0.1 | A (low | 2.2 |
| | | | | | MPa | crystallinity) | |
| 9 | 20 | g | 98.54 | 0.49 | 45 °C/24 h/−0.07 | A | 3.3 |
| | | | | | MPa | | |

### Example 11. Effect of Process Parameters on Drying Kinetics and Form D to Form F Transition

A large-scale drying profile and time-course experiment was performed using representative wet Form D freebase. To achieve adequate control of the process conditions, a jacketed agitated filter dryer was utilized. Water content of the solid samples was measured using a Karl Fischer (KF) (oven method) test, while being careful to promptly seal the materials in crimp-cap vials. To prevent unintended fluctuations in water content when determining polymorphic form, these measurements were promptly conducted after loading the solids onto the XRPD plate in a 3040 RH humidity-controlled room.

Table 11 details the progression of a typical drying process performed at a vacuum (from about −0.09 to about −0.1 MPa), with intermittent agitation (about 3-4 min/h at about 3-4 rpm). The experiment began with the Form D wet cake (~20% KF) obtained following an acetone displacement wash and deliquoring. Initial vacuum drying at 25 °C jacket temperature removed free water, reaching ~10% KF value within about 12 hours. Subsequent dehydration the removal of water from the crystal lattice—occurred slowly after the temperature was increased to 45 °C. This stage required additional 48 hours to achieve a target 4-5 KF value, successfully yielding Form F with no residual Form D remaining.

TABLE 11
Drying progression using two-stage process.

| | KF value | Polymorphic |
|---|---|---|
| Sample Point | (% water) | Form |
| Wet cake after slurrying in acetone/water | 36.6 | D |
| Wet cake after slurrying in acetone | 26.4 | D |
| Wet cake after a second acetone slurry | 20.2 | D + F |
| Drying at 25 °C for 6 h | 13.0 | D + F |
| Drying at 25 °C for 12 h | 10.5 | D + F |
| Drying at 45 °C for 4 h | 9.6 | D + F |
| Drying at 45 °C for 8 h | 10.3 | D + F |
| Drying at 45 °C for 12 h | 10.3 | D + F |
| Drying at 45 °C for 16 h | 10.0 | D + F |
| Drying at 45 °C for 20 h | 9.7 | D + F |
| Drying at 45 °C for 24 h | 9.1 | D + F |
| Drying at 45 °C for 30 h | 8.0 | D + F |
| Drying at 45 °C for 36 h | 6.8 | D + F |
| Drying at 45 °C for 42 h | 4.5 | F |
| Drying at 45 °C for 48 h | 4.4 | F |

To investigate the risk of over-drying, Form F (4.0% KF) was resubjected to vacuum (from about −0.09 to about −0.1 MPa) with continuous agitation at various jacket temperatures. As shown in Table 12, Form F remained stable at 35 °C for up to 24 hours. However, at 45 °C, the moisture content dropped below 20% within 6 hours, and conversion to Form G began after 12 hours. Although the current process operates at this higher temperature, the risk is manageable as this represents a worst-case scenario, as drying kinetics are heavily influenced by scale and equipment choice.

TABLE 12
Over-drying kinetics starting with dry Form F (initial 4.0% KF).

| Jacket temp | Drying time | Water Content | |
|---|---|---|---|
| (°C) | (h) | (oven KF) | Polymorphic Form |
| 35 | 6 | 4.4% | F |
| | 12 | 4.4% | F |
| | 18 | 4.3% | F |
| | 24 | 4.3% | F |
| 45 | 6 | 1.98% | F |
| | 12 | 1.7% | F + G |
| | 18 | 1.59% | F + G |
| | 22 | 1.25% | F + G |

### Example 12. Generating Form F by Reslurry of Form D in Dry Solvents

We present an alternate method for the preparation of sepiapterin Form F by reslurrying the intermediate Form D wet cake in a dry solvent. A challenge in using vacuum drying to control form conversion (as outlined in Example 11) is the difficulty in simultaneous and precise control of several variables, that include but are not limited to vacuum pressure, agitation rate, filter dryer dimensions and temperature, while being careful to avoid over-drying to anhydrous Form G.

A wet cake comprising Form D (from Example 3) was isolated (20-25% water content in the solids) and then reslurried in a dry solvent for time periods and jacket temperatures indicated in Table 13 and the XRPD of the wet solids was measured.

TABLE 13
Effect of re-slurry of wet 1 Form D in dry solvents

| | Water | | | | | |
|---|---|---|---|---|---|---|
| | Content | Temp | Time | Form of slurry | Physical | |
| Solvent | (KF) | (°C) | (h) | after aging time | Attributes | % LC Purity |
| EtOH | 0.03% | 25 | 20 | F | free-flowing | 98.35 |
| 1-PrOH | 0.09% | 25 | 20 | F | slurry | 98.57 |
| 2-PrOH | *nd | 25 | 20 | F | | 98.30 |
| acetone | 0.33% | 25 | 20 | F | | 98.55 |
| MeCN | 0.06% | 25 | 20 | F | | 98.47 |
| THF | 0.02% | 25 | 40 | F | | 98.53 |
| MeTHF | 0.05% | 35 | 16 | F | solids sticking | 98.49 |
| toluene | *nd | 35 | 20 | F | to walls | 98.27 |
| MIBK | *nd | 50 | 4 | F | | 97.84 |
| IPAc | 0.05% | 35 | 16 | F | | 98.56 |

*nd = not determined

Provided the overall water content of the system was maintained at an appropriate level (we found this operation to be reliable when the sum of water in the solid and incoming solvent is 2.5 wt % or less with respect to all components), form-turnover occurred readily. While water-miscible solvents enabled form turnover at 25 °C, water-immiscible solvents necessitated heating to 35-50 °C to overcome mixing inefficiencies arising from liquid-liquid biphase irregularities. This process, which did not require azeotropic water removal, could be conducted in either a vessel or a filter dryer. Experiments run with water-immiscible solvents often resulted in less desirable physical product attributes, such as solids adhering to the vessel walls.

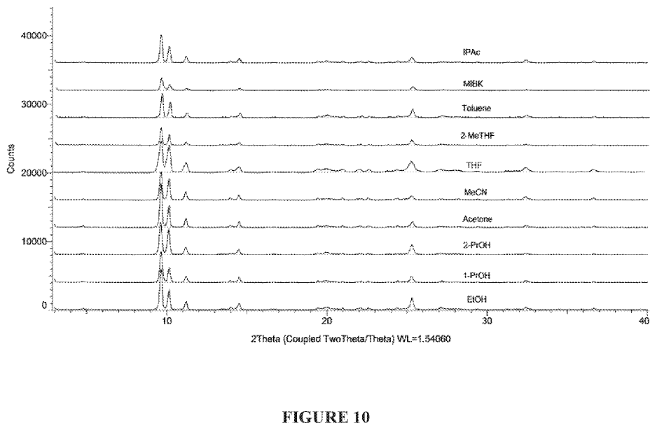

FIG. 10 provides an overlay of X-ray diffraction patterns of wet cakes obtained after reslurry of sepiapterin Form D wet cake in dry solvents.

### Example 13. Interconversion of Forms D, F and G

The following example outlines factors affecting the interconversion between crystalline freebase Forms D, F and G. Over-drying of Form F (a ¾ hydrate) can lead to the undesired formation of anhydrous Form G. This process is reversible and Form G will rapidly absorb atmospheric moisture, converting back to Form F which can potentially lead to mistaken assignment of the polymorphic form. Because of this potential pitfall, we carefully studied both the thermodynamic of the phase boundary as a function of solid KF and relative humidity as well as the kinetics for conversion from Form G to Form F.

### Rate of Hydration/Form Change for Form G Exposed to Atmospheric Moisture

Form G was prepared according to the following procedure: Form F solids were loaded onto a silicon wafer XRPD sample holder. The plate was transferred to a vacuum oven and held at 70 °C for 1.5 h under reduced pressure (from about −0.09 to about −0.1 MPa), delivering qualitatively pure Form G. The conversion of Form G to Form F under ambient conditions, with or without the use of KAPTON® film was then performed as follows:

Results without KAPTON® Film during humidity exposure: In a separate experiment, an XRPD sample holder containing sepiapterin Form G, prepared as described above, was exposed to ambient conditions (25 °C, 33%-40% RH) for 1 min, 3 min, 5 min, 7 min, and 10 min, respectively. After this period elapsed, the samples were covered with a KAPTON® film and analyzed by XRPD immediately (test time: 4 min; the humidity in the XRPD chamber: 37%-40% RH on the humidity reader).

Results with KAPTON® Film during humidity exposure: In separate experiments, an XRPD sample holder containing sepiapterin Form G, prepared as described above, was covered by KAPTON® film under ambient condition (25 °C, 310%-37% RH) and then further left at ambient condition (25 °C, 31% o-37% RH) for 1 min, 3 min, 5 min, 7 min, 10 min, 15 min, 20 min, 25 min, 30 min, 35 min, and 40 min, respectively. The samples were then immediately analyzed by XRPD (test time: 4 min; XRPD chamber RH humidity: 36%-38%). Table 14 compares the results with and without the KAPTON® film

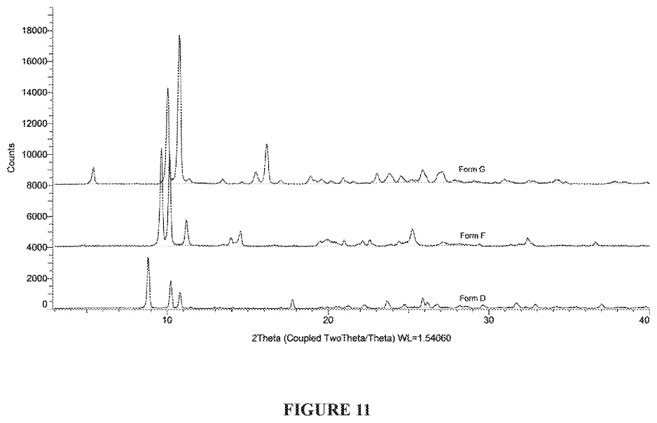

FIG. 11 provides an overlay of X-ray diffraction patterns of reference forms D, F, Gl.

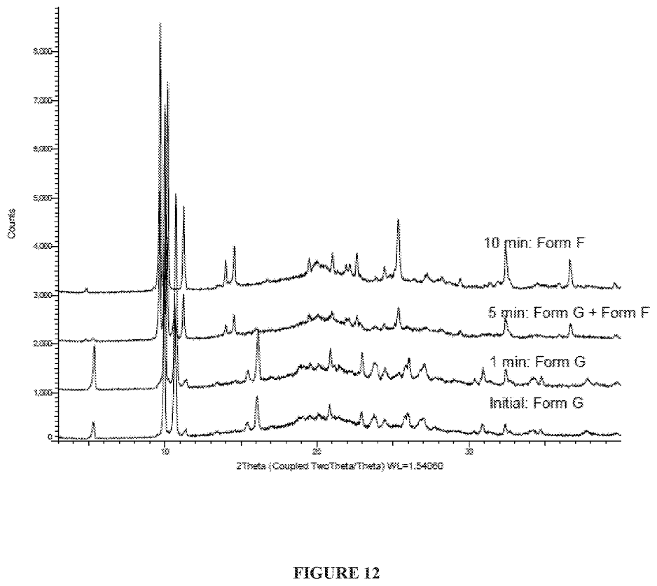

FIG. 12 provides an overlay of X-ray diffraction patterns obtained from Form stability experiments without KAPTON® film.

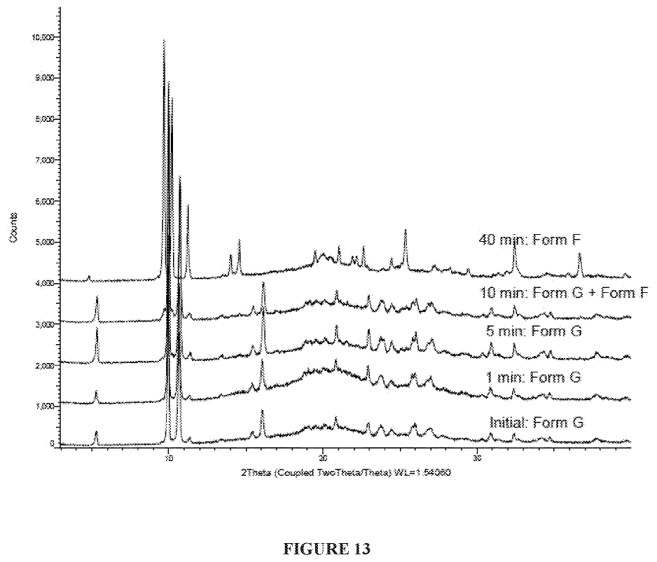

FIG. 13 provides an overlay of X-ray diffraction patterns obtained from Form stability experiments with KAPTON® film.

TABLE 14
Stability of Form G during XRPD analysis

| | Polymorphic Form | Polymorphic Form |
|---|---|---|
| | (without KAPTON ® film | (with KAPTON ® film |
| Time Point | covering XRPD sample) | covering XRPD sample) |
| Initial | G | G |
| 1 minute | G | G |
| 3 minutes | G + F | G |
| 5 minutes | G + F | G |
| 7 minutes | G + F | G |
| 10 minutes | F | G + F |
| 15 minutes | Not tested | G + F |
| 20 minutes | Not tested | G + F |
| 25 minutes | Not tested | G + F |
| 30 minutes | Not tested | G + F |
| 35 minutes | Not tested | G + F |
| 40 minutes | Not tested | F |

The data obtained for Form G stability under ambient conditions shows that its rehydration is extremely fast, even at the relatively modest humidity of 33%-40% RH, with conversion to Form F complete within 10 minutes. Therefore, if a solid sample is taken and tested for the presence of Form G, stringent control of moisture must be applied or obtaining a false negative result becomes a significant risk. There are a number of measures that can be taken such as use of a glovebox, XRPD sample dome, or KAPTON® film. When the experiments were repeated with KAPTON® film fixed in place over the solids, full conversion to Form F did not occur until between 35 and 40 minutes. In summary, this particular analysis requires close attention to detail and careful handling such that simply leaving a sample in a queue without knowing how long until data acquisition occurs is advised against.

### DVS Experiments

The effect of variable water, to precise values, on the polymorphic form of sepiapterin free base was investigated using a dynamic vapor sorption (DVS) instrument. Because of the faster rate for water absorption by Form F and faster rate of desorption from Form D (as exhibited by hysteresis in the full-cycle DVS experiment from 0% to 95% to 0% RH), two types of experiments were run. In a first experiment, the relative humidity was set to zero to obtain a baseline weight correlated with no water or 0% KF, increased to 50%, then slowly lowered to final values of 3%, 4% or 5% in separate experiments. To probe high KF values, the samples were again initially dried-out at 0% RH, then the relative humidity was slowly increased to final values of 60%, 62.5% or 65% in separate experiments. The overall weight change was recorded for each experiment. After each DVS experiments was complete, the sample pan was taken out and the contained solids were quickly spread onto the XRPD sample holder, and covered with a knife edge beam stop dome to prevent equilibration with the air. The sample holder was then immediately transferred to the XRPD instrument for data acquisition.

TABLE 15
DVS and XRPD data indicating KF
boundaries between Forms D, F, G

| | Water Content Inferred | Polymorphic |
|---|---|---|
| | by Weight Change from | Form (XRPD at |
| DVS Cycle/Method | Fully Dry Sample | end point) |
| 0% ® 50% RH ® 3% RH | 50% RH: 4.6% | G + trace F |
| dm/dt = 0.001%/min | 3% RH: 0.05% | |
| 0% ® 50% RH ® 4% RH | 50% RH: 4.2% | F + trace G |
| dm/dt = 0.001%/min | 4% RH: 3.4% | |
| 0% ® 50% RH ® 5% RH | 50% RH: 4.5% | F |
| dm/dt = 0.001%/min | 5% RH: 3.6% | |
| 0% ® 60% RH | 60% RH: 4.5% | F |
| dm/dt = 0.001%/min | | |
| 0% ® 62.5% RH | 62.5% RH: 5.0% | F + trace D |
| dm/dt = 0.002%/min | | |
| 0% RH ® 65% RH | 90% RH: 7.1% | D + trace F |
| dm/dt = 0.002%/min | | |

The data from DVS experiments and XRPD analysis is summarized in Table 15. The data demonstrates that strict control of the water content is needed, with a KF window of 3.6%-4.5%, in which Form F is present in the absence of other forms. From the standpoint of the drying process, sufficient water must be removed to convert Form D to Form F, which, in terms of KF can be expected to range between 4.5% and 5.0%.

Taken together, these two sets of experiments (Form G stability and DVS) demonstrate that while over-drying of Form F may lead to Form G, the obtained Form G may readily convert back to Form F under ambient conditions. Furthermore, Form D must be dried enough to give Form F, but not so much as afford Form G. The use of a KAPTON© film cover or other protective measure when handling samples of Form F is thus important.

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A method of preparing crystalline sepiapterin free base Form F comprising:
(a) treating sepiapterin free base with an acid to obtain sepiapterin salt, and freebasing the salt to obtain a purified sepiapterin free base;
(b) thermocycling the purified sepiapterin free base from step (a) in one or more solvents to obtain sepiapterin free base, and
(c) filtering and drying the sepiapterin free base from step (b) to obtain crystalline sepiapterin free base Form F.

2. The method of claim 1, wherein the thermocycling comprises heating to a temperature of from 25 °C to 75 °C followed by cooling to a temperature of less than 25 °C.

3. The method of claim 1, wherein the freebasing comprises adjusting pH with addition of a base selected from potassium hydroxide, potassium phosphate tribasic, potassium phosphate dibasic, sodium phosphate dibasic, sodium phosphate monobasic, sodium carbonate, potassium carbonate, sodium bicarbonate, potassium bicarbonate, and sodium hydroxide.

4. The method of claim 1, wherein the sepiapterin salt is selected from sepiapterin hydrochloride, sepiapterin mesylate, sepiapterin besylate, and sepiapterin sulfate.

5. The method of claim 1, wherein the one or more solvents in step (b) are selected from the group consisting of water, ethanol, acetone, isopropyl alcohol, 2-butanone, tetrahydrofuran, 2-methyltetrahydrofuran, N,N-dimethylformamide, N,N-dimethylacetamide, dimethyl sulfoxide, and mixtures thereof.

6. The method of claim 1, wherein the drying is maintained such that the KF value remains about 3.5% or 4%.

7. The method of claim 1, wherein freebasing comprises adjusting the pH in a range of from about pH 5 to about pH 9.

8. The method of claim 1 wherein the sepiapterin salt from step (a) comprises less than 1% of lactoylpterin, or a salt thereof.

9. The method of claim 1, wherein sepiapterin free base from step (b) does not include sepiapterin free base Form E in an amount detectable by X-ray powder diffraction.

10. The method of claim 1, wherein sepiapterin free base from step (b) includes sepiapterin free base Form D, Form F, or a mixture thereof.

11. The method of claim 1, wherein, sepiapterin free base from step (b) includes sepiapterin free base Form A, Form D, Form F, or mixtures thereof.

12. The method of claim 11, wherein the free base from step (b) is further thermocycled in one or more solvents to obtain sepiapterin free base Form D, Form F, or mixtures thereof.

13. A method of preparing crystalline sepiapterin free base form F comprising:
(a) treating sepiapterin free base with an acid to obtain a sepiapterin salt, and freebasing the salt to obtain purified sepiapterin free base;
(b) thermocycling the purified sepiapterin free base from step (a) in one or more solvents to obtain sepiapterin free base Form D, Form F, or a mixture thereof,
(c) filtering the sepiapterin free base from step (b) to obtain a wet cake comprising sepiapterin free base Form D, Form F, or a mixture thereof, and
(d) drying by reslurrying the wet cake from step (c) in a dry solvent to obtain crystalline sepiapterin free base Form F.

14. The method of claim 13, wherein the one or more solvents in step (b) are selected from the group consisting of water, ethanol, acetone, isopropyl alcohol, 2-butanone, tetrahydrofuran, 2-methyltetrahydrofuran, N,N-dimethylformamide, N,N-dimethylacetamide, dimethyl sulfoxide, and mixtures thereof.

15. The method of claim 13, wherein the dry solvent in step (d) is selected from the group consisting of ethanol, acetone, 1-propanol, isopropanol, acetonitrile, 2-butanone, methyl isobutyl ketone, isopropyl acetate, toluene, tetrahydrofuran, 2-methyltetrahydrofuran, and mixtures thereof.

16. The method of claim 13, wherein freebasing comprises adjusting the pH in a range of from about pH 5 to about pH 9.

17. The method of claim 13 wherein the sepiapterin salt from step (a) comprises less than 1% of lactoylpterin, or a salt thereof.

18. The method of claim 13, wherein sepiapterin free base from step (b) does not include sepiapterin free base Form E in an amount detectable by X-ray powder diffraction.

19. A method of preparing crystalline sepiapterin free base form F comprising:
(a) treating sepiapterin free base with an acid to obtain a sepiapterin salt, and freebasing the salt to obtain a purified sepiapterin free base; and
(b) thermocycling the purified sepiapterin free base from step (a) and drying in one or more dry solvents to obtain crystalline sepiapterin free base Form F, and optionally Form G.

20. The method of claim 19, wherein step (b) comprises one or more dry solvents selected from the group consisting of acetone, 2-butanone, methyl isobutyl ketone, isopropanol, dimethyl sulfoxide, and mixtures thereof.

21. The method of claim 19, wherein freebasing comprises adjusting the pH in a range of from about pH 5 to about pH 9.

22. The method of claim 19, wherein the sepiapterin salt from step (a) comprises less than 1% of lactoylpterin, or a salt thereof.

23. The method of claim 19, wherein sepiapterin free base from step (b) does not include sepiapterin free base Form E in an amount detectable by X-ray powder diffraction.
